(19) United States
(12) Patent Application Publication
(10) Pub. No.: US 2013/0331792 A1
(43) Pub. Date: Dec. 12, 2013

(54) DEVICE AND USES THEREOF

(71) Applicants: Jeffrey M. Karp, Brookline, MA (US); Woo Kyung Cho, Cambridge, MA (US); Bryan Laulicht, Cambridge, MA (US); James A. Ankrum, Cambridge, MA (US); Rohit N. Karnik, Cambridge, MA (US); Robert Langer, Newton, MA (US)

(72) Inventors: Jeffrey M. Karp, Brookline, MA (US); Woo Kyung Cho, Cambridge, MA (US); Bryan Laulicht, Cambridge, MA (US); James A. Ankrum, Cambridge, MA (US); Rohit N. Karnik, Cambridge, MA (US); Robert Langer, Newton, MA (US)

(73) Assignees: THE BRIGHAM AND WOMEN'S HOSPITAL, INC., Boston, MA (US); MASSACHUSETTS INSTITUTE OF TECHNOLOGY, Cambridge, MA (US)

(21) Appl. No.: 13/980,503

(22) PCT Filed: Jan. 18, 2012
(86) PCT No.: PCT/US12/21778

§ 371 Date: Aug. 20, 2013

Related U.S. Application Data
(60) Provisional application No. 61/433,934, filed on Jan. 18, 2011.
(60) Provisional application No. 61/453,521, filed on Mar. 16, 2011.

(51) Int. Cl. A61B 17/20; A61M 37/00

(52) U.S. Cl. CPC A61B 17/205; A61M 37/0015
USPC 604/174; 606/185

(57) ABSTRACT

The present disclosure provides devices and uses thereof. A devices disclosed herein comprises one or more tips, wherein the one or more tips are designed and constructed to initiate penetration by the device; and one or more protrusions in a region adjacent to each tip. In some embodiments, one or more protrusions can be constructed and arranged so that the required penetration force is reduced as compared with that observed for an otherwise identical device lacking the one or more protrusions. Additionally or alternatively, one or more protrusions can be constructed and arranged such that the required pull-out force is increased as compared with that observed for an otherwise identical device lacking the one or more protrusions.

### CROSS REFERENCES OF RELATED APPLICATIONS

The present application claims priority to U.S. provisional patent applications, U.S. Ser. No. 61/433,934, filed Jan. 18, 2011; and U.S. Ser. No. 61/453,521, filed Mar. 16, 2011, the contents of which are incorporated herein by reference.

### GOVERNMENT SUPPORT

The work described herein was supported, in part, by grants from the National Institutes of Health (grant GM086433) and the American Heart Association (grant 0835601D), the National Science Foundation (Grant NIRT 0609182) and National Institutes of Health (grant DE013023). The Government of the United States has certain rights in this application.

### BACKGROUND

The North American porcupine has ˜30,000 quills on the dorsal surface and when it encounters a predator, the release of quills is facilitated by direct contact with the predator. Each quill tip contains microscopic backward facing barbs, whereas other mammals such as the African porcupine, hedgehog, and echidna have smooth quills (or spines). If the tip of a quill strikes the skin of a predator, the resulting reaction force exerted on the shaft of the quill may be strong enough to shear the quill's root from surrounding tissue, which may help the porcupine to escape from the enemy. It has been well documented that it is difficult to remove porcupine quills from the predator once the quills are lodged within tissue (typically through both skin and muscle). However, the forces involved in penetration and pull-out have yet to be described and a comprehensive mechanism remains elusive. The biomimicry of the North American porcupine should be of interest to both scientific and industrial communities.

Although a variety of needles and their derivatives have been used, it would be beneficial to promote innovations mimicking and improving natural systems, for example, the North American porcupine's quills. More generally, there remains a need for devices and methods that facilitate penetrating and/or sticking to a substrate.

### SUMMARY

The present disclosure provides a device for penetrating a substrate and uses thereof. Such a device comprises one or more tips, wherein the one or more tips are designed and constructed to initiate penetration by the device; and one or more protrusions in a region adjacent to each tip. In some embodiments, one or more protrusions can be constructed and arranged so that the required penetration force is reduced as compared with that observed for an otherwise identical device lacking the one or more protrusions. Additionally or alternatively, one or more protrusions can be constructed and arranged such that the required pull-out force is increased as compared with that observed for an otherwise identical device lacking the one or more protrusions.

### DEFINITIONS

In order for the present disclosure to be more readily understood, certain terms are first defined below. Additional definitions for the following terms and other terms are set forth throughout the specification.

The term “biodegradable” is understood to refer to any material that it changes its chemical composition (e.g., degrades) after being placed into a live animal or live cell-containing medium, resulting in an eventual decrease in number average molecular weight.

The term “tip” as used herein typically refers to the smaller end region of an object that at least two different dimensions or a pointed region of an object that contains a projection.

The term “shaft” as used herein typically refers to a long, narrow region of an object.

The term “needle” as used herein typically refers to an object that pierces a substrate.

The term “microneedle” as used herein typically refers to a sharp object with at least one dimension on the order of 10 nanometers to 1,000 microns.

The term “Hypodermic needle” is understood in the art to refer to objects (whether solid, or containing one or a plurality of lumens) that is adapted for and capable of penetrating the epidermis of any species.

The term “barb” as used herein refers to an object that has at least one sharp pointed region that is affixed to a main body such as a shaft.

### BRIEF DESCRIPTION OF THE DRAWING

The drawing is for illustration purposes only, not for limitation.

FIG. 1 depicts exemplary deployable barbed quill array: A) quill mimetic array of barbed, in-plane needles; B) barbed needles bent out of plane prior to tissue insertion.

FIG. 2 depicts exemplary barbed hypodermic needle: A) unmodified hypodermic needle. B) barbed hypodermic needle.

FIG. 3 depicts exemplary deployable barbed microneedle array design: barbed pyramidal microneedle. In the presence of sacrificial or water-soluble components, the barb is deployable upon insertion into aqueous environments (e.g. tissues).

FIG. 4 shows: (A) Digital photograph of representative quills with different lengths of barbed regions where the length is typically in the range of 3-5 mm. (B) Optical microscopic image confirms the length of a quill with a 4 mm barbed region. (C) Sequential FE-SEM images of a single quill show the transition from functional barbs to a smooth surface containing barbs that have yet to emerge (i.e. those that cannot yet engage tissue).

FIG. 5 shows: (A-B) Digital photographs show similar diameters for (A) a porcupine quill (A) and (B) a 18 gauge needle. The small squares indicate the region that was used to measure the diameters of the quill or a needle. (C) Representative force-extension curve to show both penetration and pull-out profile of a 18 gauge needle with muscle tissue.

FIG. 6 shows: (A) FE-SEM image to show the surface feature of the African porcupine quill. (B) Representative force versus extension plot shows the penetration and removal forces for an African porcupine quill inserted into muscle tissue. (C) The table summarizes the values obtained from the processes of penetration/removal of African porcupine quill within muscle tissue (n=5). The averaged values are shown with standard deviation.

FIG. 7 shows: (A) Initial geometry of two-barbed quill with the dimensions of a single barb and the distance between two barbs indicated. (B) Finite element mesh used in the simulation. It contains both the quill and tissue, and is a snapshot from a simulation.

FIG. 8 shows: (A) Dimensional analysis of the porcupine quill for finite element analysis (FEA) through length scale measurements of natural quills (n=5). The averaged values are shown with standard deviation. In terms of curvature, there are three transition points. L and W indicate length and width, respectively. (B) Full mesh of barbless quill prior to insertion into the modeled tissue. (C) Magnified view of the fine mesh at the quill/tissue interface.

FIG. 9 shows: (A)-(H) Representative optical micrographic images confirm the barbed region for eight quills that have been sanded to obtain barbed regions of specific length. Inset in (C) shows the enlarged images for 1 mm barbed region.

FIG. 10 shows: (A) and (B) The characteristic FE-SEM images following removal of a barbed quill following a 4 mm penetration depth into tissue (For the FE-SEM image showing the quill prior to penetration into tissue, see FIG. 1B). Residual tissue was present along the length of the barbs and under the barbs as indicated with white arrows. Scale bar represents 50 μm. (C) Representative force-extension curves show penetration and pull-out forces that were obtained with fibrous muscle tissue and a density matched non-fibrous model tissue fabricated from gelatin gel (n=5).

FIG. 11 depicts representative FE-SEM images of a barbless quill following penetration-retraction tests with muscle tissue. (A) Before penetration into tissue (B) After removal from the tissue. The white arrow indicates adhered tissue due to friction between the barbless quill and the tissue.

FIG. 12 depicts representative stress versus strain curve for porcine skin through uniaxial tension tests (n=5 for each experiment). The curve is characterized by a low-stiffness region at 0˜75% strain followed by a dramatic increase as the strain increases beyond 75%. Within the low-stiffness region, the tissue fibers align in parallel until a maximum stretch point. Once the locking stretch has been reached, further extension of skin requires extension of tissue fibers, producing a huge increase in stiffness. The rubbery modulus and network interlocking stretch of porcine skin were 0.05˜0.28 MPa and 1.27˜2.35, respectively. The failure strength of porcine skin was 8.2˜15.4 MPa, which is similar to the previously reported values. It is comparable with the values of 5 to 30 MPa for human skin. The ultimate strain for porcine skin ranges from 25 to 118%, which is also similar to values of 35 to 115% for human skin.

FIG. 13 depicts representative force versus extension plots from the penetration-retraction tests of the natural quill and replica molded PU quills with muscle tissue. The work of removal for natural quill and PU non-deployable barbed quill are 0.144±0.048 mJ and 0.053±0.023 mJ, respectively.

FIG. 14 depicts (A) Representative stress versus strain curve for the North American porcupine quill obtained through uniaxial tension tests (n=5 for each experiment). Young's modulus of the tip and base were 3.25±0.24 GPa and 2.44±0.19 GPa, respectively whereas the tensile strengths were 136.54±18.46 MPa and 104.25±18.80 MPa for the tip and base, respectively. The toughness was 0.92±0.62 mJ and 54.73±35.02 mJ for tip and base, respectively, emphasizing that the quill bases were significantly more compliant than the tips. Herein, data are presented as the averaged values with standard deviation. (B) and (C) Digital photos show the base and tip of the quills before and after measuring the Young's modulus and tensile strength.

FIG. 15 shows two plots: (A) The plot shows critical load for buckling (under uniaxial load (MTest, 100 mm/min)) vs the slenderness ratio L/r (length over radius of the sample) in a dot plot (n=28) with a fitted curve shown as a red solid line. (B) L/r histogram and the cumulative distribution of quill slenderness ratios (n=101). The puncture force for pig skin obtained from penetration-retraction tests is given below the plot with the percentage of quills that puncture pig skin without buckling.

FIG. 16 is a cartoon to depict the puncture and penetration of the porcupine quill into tissue. The initial puncture is followed by penetration into the tissue. The definition of puncture force and penetration force used in this work is shown as double-pointed arrows.

FIG. 17 shows (A) Digital photograph illustrates where the quill was cut to obtain the four sections where (B)-(E), show the FE-SEM images for each section. For each low magnification image (left), high magnification representative images of the bulk of the quill (right) are shown in (F)-(I). The scale bars represent 200 μm and 20 μm for (B)-(E) and (F)-(I), respectively.

FIG. 18 shows (A) Digital photograph shows the positions of a longitudinally cut tip examined with FE-SEM. (B)-(D) low magnification FE-SEM images. The scale bars represent 200 μm. (E)-(G) high magnification FE-SEM images with scale bars representing 20 μm.

FIG. 19 shows (A) Digital photograph of base cut longitudinally. Representative areas within the bulk of the quill are indicated as a red circle and shown as (B)-(E) FE-SEM images. The center of the quill in (B) is indicated as white dotted line. The characteristic regions include (C) center, (D) boundary, and (E) edge

FIG. 20 shows amino acid compositions of the porcupine quill tip and base. The data represents the average values with standard deviation obtained from 3 quills. Error bars represent standard deviation. *Mole % is calculated based on the analyzed amino acids only.

FIG. 21 shows (A) Digital photo of the North American porcupine quill. (B) and (C) FE-SEM images showing the microstructure of the quill tip and base, respectively. (D) Fluorescence image enables visual delineation of the geometry of single barbs. (E) FE-SEM image showing the micro-structural tip of the North American porcupine quill. Scale bar represents 200 μm. (F) Representative force versus extension plots shows puncture (that occurs following 1-2 mm compression of tissue) followed by penetration and removal of barbed and barbless quills within muscle tissue. The penetrating depth of the quill was maintained at 10 mm. Inset shows the micron-level topography of barbless and barbed quills. Scale bars represent 100 μm. Arrows indicate periodic resistance encountered as the quill is removed from the tissue (this was not observed for the barbless quill). (G) Table summarizes the obtained experimental values from the processes of penetration/removal of barbed quill, barbless quill, and 18 gauge needle (n=5). The ± sign represents the standard deviation.

FIG. 22 shows (A) Absorbed strain energies in quills (barbless quills and quills with two overlapping barbs) and tissue. The energies are from finite element modeling of barbless and two-barbed quills penetrating into skin. The enlarged strain energies in quills are also shown. (B and C) Strain field distribution in skin tissue when the barbless or two-barbed quill is penetrated into the tissue. (D and E) Strain field developed in the tissue by the quill with stiffness of (D) 1000 GPa and (E) 0.001 GPa. Two simulations are identical in geometry. The stiff quill cannot penetrate as far due to non-convergence in the solution. (F) and (G) FE-SEM images show the synthetic PU quills by replica molding of natural barbed and barbless quills. The scale bar represents 100 μm. (H) The forces required to penetrate the PU quills into muscle tissue with 4 mm-depth. The averaged values are shown with standard deviation (n=5, student t-test at the level of 95% significance). The box plot whiskers are set to ±1.2 standard deviations. (I) A representative digital photo of the fabricated quill-mimetic needle. (J) The forces required to penetrate the fabricated barbed/barbless needles into a model of human skin. The data shows the averaged penetration force with standard deviation (n=3, each needle was used at least 4 times, student t-test at the level of 95% significance). The “x” below and above the whiskers of box plots in (H) and (J) indicates 1st and 99th percentile, respectively.

FIG. 23 shows (A) Finite element modeling of the quill penetration into skin tissue shows compressive stresses (in MPa) from the tissue acting on the quill at a distance from the quill tip. This is the stress state at the end of 10 mm-penetration into skin tissue. (B) and (C) Penetration and pull-out forces obtained with the prepared quills (from n=5 different quills for each preparation). The penetrating depth for all experiments was 10 mm. The averaged values are shown with standard deviation. Cartoons depict quills prepared with specific lengths of barbs obtained through ablation with sand paper. The blue color indicates the barbed region and the white color indicates the barbless region. The penetration and pull-out forces of some of the prepared quills are compared to those of the barbless quill (quill 1). The difference in force is defined as Δᵢⱼ (Δᵢⱼ=penetration (or pull-out) force of quill j-penetration (or pull-out) force of quill i)).

FIG. 24 shows (A) and (B) Representative optical and fluorescent images of porcupine quills before and after removal from porcine skin. Fluorescence images were obtained by merging several images taken at different focal planes along the Z axis. The scale bar represents 100 μm. (C)-(F) FE-SEM micrographs following removal of quills from porcine skin. Residual tissue is indicated by blue arrows. The red arrow in figures indicates bending of barbs during pull-out. (G) A representative force versus extension plot for a penetrating depth of 4 mm where puncture typically occurs following tissue compression of 1-2 mm as indicated by an arrow (n=5). (H)-(J) Optical images of quills following removal from porcine skin are useful to examine the heterogeneity of tissue interactions and to establish the relationship between the bending of barbs and relative level of tissue adhesion summarized in the table (K). The scale bar in each image represents 100 μm. (L) The digital photograph shows the fabricated quill-mimetic patch, which consists of 7 PU barbless or barbed quills. (M) The tissue adhesion forces obtained from barbless and barbed PU quill patches (n=5, student t-test at the level of 95% significance). The box plot whiskers are set to ±1.2 standard deviations. (N) The digital photographs show the quill-mimetic patches interacting with muscle tissue during the retraction process from muscle tissue

### DETAILED DESCRIPTION OF CERTAIN EMBODIMENTS

A device for penetrating a substrate used in accordance with the present disclosure, in theory, can be of any shape or design. For example, a device or a body of the device can be or comprise a film, a sheet, a tape, a needle, an array, a hook, and/or a probe.

A tip of a device, in general, refers to an end and/or pointed region of an object that is sufficiently sharp to initiate penetration. Typically, a tip is an extremity of something slender or tapering and may contain a shaft of any shape (e.g., a tapered region) that connects with a body of a device used in accordance with the present disclosure. A device may contain one or more tips.

Each tip may independently have one or more protrusions in a region adjacent to the apex of tip. Such one or more protrusions can be constructed and arranged so that the required penetration force is reduced as compared with that observed for an otherwise identical device lacking the one or more protrusions. In some embodiments, a device described herein can be characterized by the required penetration force being reduced to or less than 5%, 10%, 15%, 20%, 25%, 30%, 35%, 40%, 50%, 75% or 90%, as compared with that observed for an otherwise identical device lacking the one or more protrusions. In some embodiments, a device described herein can be characterized by the required penetration force being reduced to in a range of any two values above, as compared with that observed for an otherwise identical device lacking the one or more protrusions. In addition or alternatively, such one or more protrusions can be constructed and arranged such that the required pull-out force is increased as compared with that observed for an otherwise identical device lacking the one or more protrusions. In some embodiments, a device described herein can be characterized by the required pull-out force being increased to or more than 1500%, 1000%, 500%, 400%, 300%, 250%, 200%, 150%, or 125%, as compared with that observed for an otherwise identical device lacking the one or more protrusions. In some embodiments, a device described herein can be characterized by the required pull-out force being increased to in a range of any two values above, as compared with that observed for an otherwise identical device lacking the one or more protrusions. In some embodiments, the diameter of the penetration point of each tip can be less than 140%, 120%, 110% or 105% of that observed for an otherwise identical device lacking the one or more protrusions.

In some embodiments, intended for biological applications, dimensions or dimensional ratios of a device are on the order of those exhibited by North American porcupine quills. In some embodiments, dimensions or ratios may be significantly smaller or larger than those of the quills, even by orders of magnitude.

Protrusions

In general, devices described herein comprises one or more tips and one or more protrusions extending from the tip surface, in a region adjacent to each tip.

In certain embodiments, devices described herein may comprise a single protrusions. In various embodiments, a plurality of protrusions includes two or more protrusions. In some embodiments, the number of protrusions can be up to the order of a billion. Typically, the number of protrusions utilized in a particular device may depend on the spacing and the area having protrusions protrude from. For example, protrusions can be spaced from one another about or less than 1 cm, 5 mm, 1 mm, 500 microns, 200 microns, 100 microns, 50 microns, or 10 microns. A spacing between protrusions can be in a range of 1 cm to 5 mm, 5 mm to 1 mm, 500 microns to 200 microns, 200 microns to 100 microns, 100 microns to 50 microns, 50 microns to 10 microns, 10 microns to 1 micron, or between any two values above.

In accordance with the present disclosure, a device can be arranged and constructed so that one or more protrusions protrude from the a region adjacent to each of one or more tips in different directions in three-dimensional space. For example, protrusions can protrude radially from the surface of a region adjacent to a tip, each independently having at an angle relative to the tangent to the surface or relative to a shaft from which it protrudes. In some embodiments, protrusions are projected outward in the opposite direction of the tip that the protrusions are adjacent to. Each protrusion of the plurality can independently have an angle of 90 degrees or any others less than 90 degrees. In some embodiments, such an angle can be about or less than 80 degrees, 70 degrees, 60 degrees, 50 degrees, 40 degrees, 30 degrees, 20 degrees, 10 degrees, 5 degrees, 4 degrees, 3 degrees, 2 degrees, 1 degree or even 0 degree. In some embodiments, such an angle can be in a range of 0-90 degrees, 1-60 degrees, 1-50 degrees, 1-30 degrees, 1-20 degrees, 1-10 degrees, 1-5 degrees, 1-3 degrees, or 1-2 degrees. In certain embodiments, protrusions can be unidirectional. In certain embodiments, protrusions (e.g., pyramidal protrusions) may not be directed inward or outward.

In some embodiments, the dimensions and/or shape of a protrusion, or protrusions of a plurality thereof, are designed for the particular way in which a device is to be used. Without wishing to be bound by any particular theory, parameters such as the dimensions of an individual protrusion (e.g., a length, width, thickness), and/or the shape of the protrusion (e.g., barb-shaped, etc.) may influence the penetration and/or pull-out of the tip where the protrusion is placed adjacently to, and thus the efficiency and functions of the device.

Dimensions of a protrusion generally includes a length, a width and a thickness. In some embodiments, a protrusion is barb-shaped or in any other shape, and can has a maximum width. Protrusions can be designed in different shapes independently depending on applications. In general, shapes that locally maximize stress concentrations at fine points around the periphery can be used in accordance with the present disclosure as good cutting shapes with reduced insertion force. Shapes that spread the tissue around larger features will help raise removal force. For example, a protrusion may be barb-shaped, hemisphere, pyramid, harpoon-shaped, triangle, conical, hook-shaped, oval or Y-shaped.

In some embodiments, at least one dimension of an individual protrusion may be about or less than 1 cm, 5 mm, 2 mm, 1 mm, 500 μm, 300 μm, 250 μm, 200 μm, 150 μm, 120 μm, 100 μm, 90 μm, 80 μm, 70 μm, 60 μm, 50 μm, 40 μm, 30 μm, 20 μm, 10 μm, 5 μm, 1 μm, or even 500 nm. In some embodiments, the length of an individual protrusion may be more than 500 nm, 1 μm, 5 μm, 10 μm, 20 μm, 30 μm, 40 μm, 50 μm, 60 μm, 70 μm, 80 μm, 90 μm, 100 μm, 120 μm, 150 μm, 200 μm, 250 μm, 300 μm, 500 μm, 1 mm, 2 mm, 5 mm, or even 1 cm. In some embodiments, at least one dimension of an individual protrusion may be in a range of 1 cm to about 1 μm. In some embodiments, at least one dimension of an individual protrusion may be in a range of 1 mm to 10 μm. In some embodiments, at least one dimension of an individual protrusion may be in a range of 500 μm to 100 μm. In some embodiments, at least one dimension of an individual protrusion may be in a range of 200 μm to 100 μm. In some embodiments, at least one dimension of an individual protrusion may be in a range of 120 μm to 100 μm. In some embodiments, at least one dimension of an individual protrusion may in a range of 70 μm to about 50 μm. In some embodiments, at least one dimension of an individual protrusion may be in a range of 50 μm to 10 μm. In some embodiments, at least one dimension of an individual protrusion may be in a range of any two values above. It may be desirable, in certain embodiments, to adjust at least one dimension of a protrusion according to the application/use of the device.

In some embodiments, an aspect ratio of one dimension to another dimension (e.g., length/width) of an individual protrusion may be about, less than or more than 50, 20, 10, 9, 8, 7, 6, 5, 4, 3, 2, 1, 0.5, 0.2, or even 0.1. In some embodiments, an aspect ratio of one dimension to another dimension may be in a range of 50-1. In some embodiments, an aspect ratio of one dimension to another dimension may be in a range of 10-1. In some embodiments, an aspect ratio of one dimension to another dimension may be in a range of 5-1. In some embodiments, an aspect ratio of one dimension to another dimension may be in a range of 2-1. In some embodiments, an aspect ratio of one dimension to another dimension may be in a range of any two values above. It may be desirable, in certain embodiments, to adjust an aspect ratio of one dimension to another dimension of a protrusion according to the application/use of the device.

In some embodiments, protrusions may overlap with one another. In some embodiments, protrusions can have an overlap of about, less than or more than 1%, 5%, 10%, 20%, 30%, 40%, 50%, 60%, 70%, 80% or even 90% of the protrusion size. In some embodiments, an overlap may be in a range of 1-50% of the protrusion size. In some embodiments, an overlap may be in a range of 5-30% of the protrusion size. In some embodiments, an overlap may be in a range of 10-20% of the protrusion size. In some embodiments, an overlap may be in a range of any two values above of the protrusion size. Without being bound by any particular theory, it is proposed that in some embodiments, when protrusions have overlapped features, they may affect the functions of a device cooperatively. It may be desirable, in certain embodiments, to adjust an overlap according to the application/use of the device.

As noted above, a tip described herein may include a shaft that connects with a body of a device. Typical shafts have a tapered region. For example, a shaft can a tapered column, cone, pyramid, hemisphere, or triangle. The dimension of a cross section of a tip, typically on its shaft, may vary depending on the design/use of a device used in accordance with the present disclosure. In some embodiments, the dimension of a cross section may be about, less than or more than 10 cm, 5 cm, 4 cm, 3 cm, 2 cm, 1 cm, 5 mm, 1 mm, or even 500 lam. In some embodiments, the dimension of a cross section may be in a range of 1 cm and 1 mm. In some embodiments, the dimension of a cross section may be in a range of any two values above.

Protrusions in accordance with the present disclosure can be arranged and constructed to protrude from a region adjacent to each tip. In some embodiments, protrusions are located in a tapered region adjacent to each tip. An adjacent region depending on design and application can be in a distance away from the apex of a tip. For example, to mimic a porcupine quill as illustrated in Example below, the distance may be a relative distance observed from a porcupine quill. In some embodiments, an adjacent region is about, less than or more than 0.01 mm, 0.1 mm, 0.5 mm, 1 mm, 2 mm, 3 mm, 5 mm, 6 mm, 7 mm, 8 mm, 9 mm, 1 cm, 2 cm, 4 cm, 5 cm, or even 10 cm away from the apex of a tip. In some embodiments, an adjacent region may be in a range of 0-10 mm, 1-5 mm, 0-2 mm, 2-4 mm, or 3-4 mm away from the apex of a tip. In some embodiments, an adjacent region may be in a range of any two values above away from the apex of a tip. Depending on the size/use of a device, a distance away from the apex of a tip can be adjusted and relative to the dimension of the cross section of the tip's shaft where protrusions locate. For example, a distance away from the apex of a tip can be about or less than 0.1 fold, 0.5 fold, 1 fold, 2 fold, 5 fold, 10 fold, or even 20 fold of the dimension of a cross section. Without being bound to any particular theory, protrusions located near the apex of a tip may exhibit a great impact on required pull-out force of a device while protrusions located next to and away from the tip apex may exhibit substantial impact on minimizing the required penetration force.

In addition to the above discussion on protrusion dimensions, in some embodiments, at least one dimension of an individual protrusion can be adjusted and relative to the dimension of the cross section of the tip's shaft where protrusions locate. For example, at least one dimension of an individual protrusion can be about or less than 0.1 fold, 0.5 fold, 1 fold, 2 fold, 5 fold, 10 fold, or even 20 fold of the dimension of a cross section.

Materials

A device including one or more tips and one or more protrusions as described herein can be made of or comprise one or more materials. Different portions can be made of or comprise different materials for different properties. For example, a device may have a body that is made of or comprising a non-swelling material. A body of a device can be anti-adhesive or repellant. Additionally or alternatively, a body of a device can be non-erodible or non-degradable.

Exemplary materials include, but are not limited to, metals (e.g., gold, silver, platinum, steel or other alloys); metal-coated materials; metal oxides; plastics; ceramics; silicon; glasses; mica; graphite; hydrogels; and polymers such as non-degradable or biodegradable polymers; and combinations thereof. In general, materials can be utilized in any form and/or for different purposes and/or in different regions (e.g., one or more tips and their adjacent regions).

Without being bound to any particular theory, varying compositions of materials used in accordance with the present disclosure (e.g., components, weight percentages, molecular weight, etc.) can affect properties of the materials for different functions/applications. For example, a substrate that a device penetrates into can be compliant and one or more tips/protrusions according to the present disclosure can be made and characterized by a stiffness being greater than that of the substrate. In certain embodiments, the stiffness of tips/protrusions can be about or more than 2 fold, 5 fold, 10 fold, 20 fold, 30 fold, 40 fold, 50 fold, or 100 fold of that of the substrate.

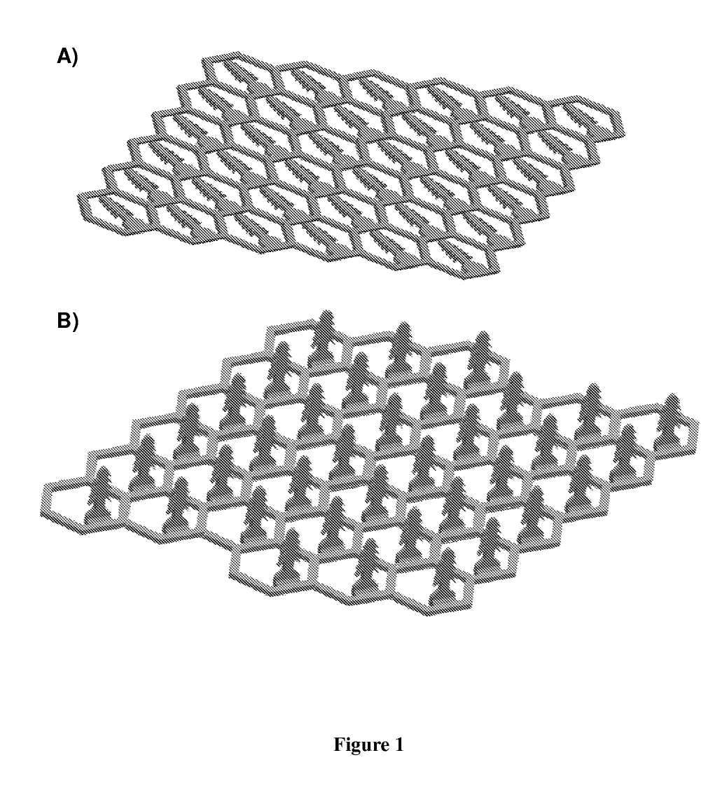

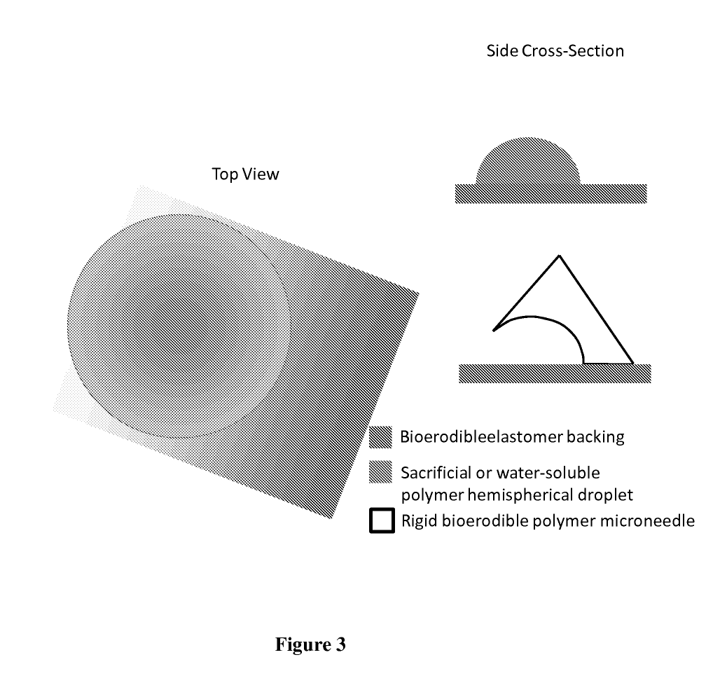

In some embodiments, a device can be made of or comprise deformable materials. As an example, a portion of a device (e.g., one or more protrusions) can be made of or comprise a pliable polymer, which may have a low bending modulus. A deployable protrusion may be able to deploy or bend and the deployment/bending may affect penetration and/or pull-out of the device. Exemplary deployable protrusions are illustrated in FIGS. 1 and 3. It may be desirable, in certain embodiments, to adjust, for example, the molecular weight of a polymer, or the weight percentages of metals in an alloy, to achieve a certain bending ability.

To give another example, a deformable material (e.g., hydrogels, thermoplastics, shape memory materials) can change shape/size depending on pressure or temperature, and can be used in different portions of a device. In certain embodiments, one or more tips of a device can be made of or contain a water-swellable material (e.g., hydrogel). In certain embodiments, one or more protrusions of a device can be made of or contain a shape memory material. Shape memory materials can change to a trained shape in response to an activation signal. Exemplary shape memory materials include, but not limited shape memory alloys (SMA) and shape memory polymers (SMP), as well as shape memory ceramics, electroactive polymers (EAP), ferromagnetic SMAs, electrorheological (ER) compositions, magnetorheological (MR) compositions, dielectric elastomers, ionic polymer metal composites (IPMC), piezoelectric polymers, piezoelectric ceramics, and various combinations thereof. Suitable shape memory alloy materials include, without limitation, nickel-titanium based alloys, indium-titanium based alloys, nickel-aluminum based alloys, nickel-gallium based alloys, copper based alloys (e.g., copper-zinc alloys, copper-aluminum alloys, copper-gold, and copper-tin alloys), gold-cadmium based alloys, silver-cadmium based alloys, indium-cadmium based alloys, manganese-copper based alloys, iron-platinum based alloys, iron-platinum based alloys, iron-palladium based alloys, and the like. Alloys can be binary, ternary, or any higher order so long as the alloy composition exhibits a shape memory effect, e.g., change in shape orientation, damping capacity, and the like. More discussion of shape memory materials can be found in US Patent Application US 20090241537, the contents of which are incorporated by references. In certain embodiments, a shape memory materials used in accordance with the present disclosure is nitinol. Without being bound to any particular theory, a deformable protrusion utilizing a shape memory material, upon increasing temperature by insertion into a substrate, can deploy by reverting back to a shape memory annealed form.

In some embodiments, a device can be made of or comprise adhesive materials (e.g., adhesive polymers). As examples, bioadhesives such as chitosan and carbopol can be used. Use of an adhesive material may be beneficial in penetrating and retaining in a substrate.

In some embodiments, a device can be made of or comprise erodible and/or degradable materials. To give an example, a tip may contain a shaft that can degrade to release the very pointed tip portion from the device body. In addition or alternatively, a protrusion can be detached from a tip after penetration, and it may remain in a substrate or erode/degrade so that the required penetration force of the device can be achieved without increased pull-out force. In certain embodiments, tips and/or protrusions can be erodible and/or degradable and a device body can degrade/erode more slowly that the tips or protrusions. Erodible and/or degradable materials can be used to coat a device or any portion of it described herein.

In some embodiments, a device can be made of or comprise one or more polymers. For example, a portion of the device (e.g., tips and/or protrusions in a region adjacent to tips) as discussed below used in accordance with the present disclosure can be made of or comprise one or more polymers. Various polymers and methods known in the art can be used. Polymers may be natural polymers or unnatural (e.g. synthetic) polymers. In some embodiments, polymers can be linear or branched polymers. In some embodiments, polymers can be dendrimers. Polymers may be homopolymers or copolymers comprising two or more monomers. In terms of sequence, copolymers may be block copolymers, graft copolymers, random copolymers, blends, mixtures, and/or adducts of any of the foregoing and other polymers.

A polymer used in accordance with the present application can have a wide range of molecular weights. In some embodiments, the molecular weight of a polymer is greater than 5 kDa. In some embodiments, the molecular weight of a polymer is greater than 10 kDa. In some embodiments, the molecular weight of a polymer is greater than 50 kDa. In some embodiments, the molecular weight of a polymer ranges from about 5 kDa to about 100 kDa. In some embodiments, the molecular weight of a polymer ranges from about 10 kDa to 50 kDa.

In some embodiments, polymers may be synthetic polymers, including, but not limited to, polyethylenes, polycarbonates (e.g. poly(1,3-dioxan-2-one)), polyanhydrides (e.g. poly(sebacic anhydride)), polyhydroxyacids (e.g. poly(β-hydroxyalkanoate)), polypropylfumarates, polycaprolactones, polyamides (e.g. polycaprolactam), polyacetals, polyethers, polyesters (e.g. polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines and copolymers thereof. In some embodiments, polymers include polymers which have been approved for use in humans by the U.S. Food and Drug Administration (FDA) under 21 C.F.R. §177.2600, including, but not limited to, polyesters (e.g. polylactic acid, poly(lactic-co-glycolic acid), polycaprolactone, polyvalerolactone, poly(1,3-dioxan-2-one)); polyanhydrides (e.g. poly(sebacic anhydride)); polyethers (e.g., polyethylene glycol); polyurethanes; polymethacrylates; polyacrylates; polycyanoacrylates; copolymers of PEG and poly(ethylene oxide) (PEO).

PEGs may be useful, in some embodiments, in accordance with the present application since they are nontoxic, non-immunogenic, inert to most biological molecules (e.g. proteins), and approved by the FDA for various clinical uses. PEG polymers can be covalently crosslinked using a variety of methods to form hydrogels. In some embodiments, PEG chains are crosslinked through photopolymerization using acrylate-terminated PEG monomers. In addition to chemical modification, block copolymers of PEG, such as triblock copolymers of PEO and polypropylene oxide) (henceforth designated as PEO-b-PPO-b-PEO), degradable PEO, poly(lactic acid) (PLA), and other similar materials, can be used to add specific properties to the PEG.

In some embodiments, polymers used herein can be a degradable polymer. Such a degradable polymer can be hydrolytically degradable, biodegradable, thermally degradable, and/or photolytically degradable polyelectrolytes.

Degradable polymers known in the art, include, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly (beta-amino esters), which may be suitable for use in accordance with the present application.

Suitable degradable polymers, and derivatives or combinations thereof, as discussed above can be selected and adapted to have a desired degradation rate. Alternatively or additionally, a degradation rate may be fine-tuned by associating or mixing other materials as previously described (e.g., non-degradable materials) with one or more of degradable polymers.

In general, a degradation rate as used herein can be dictated by the time in which a material degrades a certain percentage (e.g., 50%) in a certain condition (e.g., in physiological conditions). In some embodiments, the degradation time of a device or a portion of the device as described herein can have a wide range. In some embodiments, the degradation time may be greater than 1 minute, 5 minutes, 30 minutes, 1 hour, 2 hours. 5 hours, 12 hours, 24 hours, 1.5 days, 2 days, 5 days, 7 days, 15 days, 30 days, 2 months, 6 months, 1 year, 2 years, or even 5 years. In embodiments, the degradation time may be about or less than 10 years, 5 years, 2 years, 1 year, 6 months, 2 months, 30 days, 15 days, 7 days, 5 days, 2 days, 1.5 days, 24 hours, 12 hours, 5 hours, 2 hours, 1 hour, 30 minutes or even 5 minutes. The degradation time may be in a range of 12-24 hours, 1-6 months, or 1-5 years. In some embodiments, the degradation time may be in a range of any two values above.

In addition or alternatively, suitable shape memory polymers as mentioned above include thermoplastics, thermosets, interpenetrating networks, semi-interpenetrating networks, or mixed networks. The polymers can be linear or branched thermoplastic elastomers with side chains or dendritic structural elements. Suitable polymer components to form a shape memory polymer include, but are not limited to, polyphosphazenes, poly(vinyl alcohols), polyamides, polyester amides, poly(amino acid)s, polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyesters, polylactides, polyglycolides, polysiloxanes, polyurethanes, polyethers, polyether amides, polyether esters, and copolymers thereof. Examples of suitable polyacrylates include poly(methyl methacrylate), poly(ethyl methacrylate), ply(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Examples of other suitable polymers include polystyrene, polypropylene, polyvinyl phenol, polyvinylpyrrolidone, chlorinated polybutylene, poly(octadecyl vinyl ether) ethylene vinyl acetate, polyethylene, poly(ethylene oxide)-poly(ethylene terephthalate), polyethylene/nylon (graft copolymer), polycaprolactones-polyamide (block copolymer), poly(caprolactone) dimethacrylate-n-butyl acrylate, poly(norbornyl-polyhedral oligomeric silsequioxane), polyvinylchloride, urethane/butadiene copolymers, polyurethane block copolymers, styrene-butadiene-styrene block copolymers, and the like. In some embodiments, some multiblock copolymers that are made of or comprise (1) methylenebis(4-phenylisocyanate)/1,4-butanediol and poly(∈-caprolactone), (2) poly(ethylene terephthalate) and poly(ethylene oxide), (3) poly(2-methyl-2-oxazoline) and poly(tetrahydrofuran), (4) methylenebis(4-phenylisocyanate)/1,4-butanediol and poly(tetrahydrofuran), (5) methylenebis(4-phenylisocyanate)/1,4-butanediol and poly(ethylene adipate), (6) carbodiimide-modified diisocyanates and poly(butylene adipate), (7) ethylene glycol and poly(tetrahydrofuran) or any combination thereof.

Making and Uses

Devices in accordance with the present disclosure can be made using exemplary materials as discussed above and by suitable methods. For example, a device or any portion of it (e.g., protrusions) may be fabricated by a technique including, but not limited to, laser cutting, dry etching, wet etching, imprint coating, molding, stamping, embossing, two-photon lithography, three dimensional printing, electrospinning, imprinting, interference lithography and any combination thereof.

An example of a device or a portion of a device (e.g., a tip) suitable for use in accordance with the present disclosure can be or comprise a hypodermic needle. A tip can have at least one hole and at least one lumen. Such a hole can be used to communicate between a lumen and an exterior.

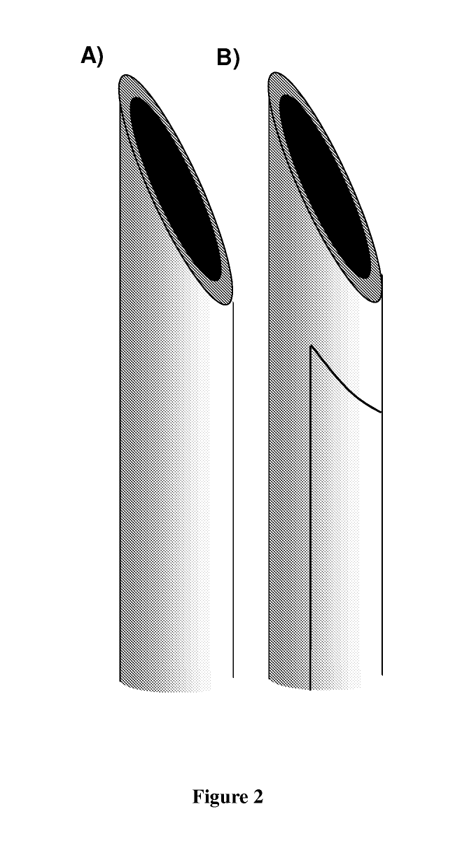

An exemplary hypodermic needle with one or more barb-shaped protrusions is illustrated in FIG. 2. Modified needles allow for a soft material such as biological tissue to infiltrate spaces in the needle shaft that then mechanically interlock with the unidirectional protrusions to increase pull-out force. A space created by the modification of the shaft can be filled with a degradable, water-soluble, or environmentally cued material that remains intact during insertion and is absent upon pull-out creating a deployable system. In certain embodiments, protrusions can be recessed at a distance from the tip of the needle that mimics the relative distance observed for a porcupine quill (e.g., 3-4 mm). In certain embodiments, protrusions can be located on the tapered portion of each tip. In certain embodiments, the bending stiffness of protrusions is on the order of that of a substrate or greater such that the protrusions bend to deploy during pull-out.

In some embodiments, a hypodermic needle is laser cut, machined, or etched to create protrusions. For example, hypodermic needles can be prepared to remove material from the shaft to create hooked needles. A hook can be covered with a sacrificial polymer layer that is present upon insertion and absent during pull-out to increase mechanical interlocking.

In some embodiments, to create raised features on the surface of a device, holes of any shape can be laser cut, machined, or etched into a hypodermic needle. A liquid material can then be introduced in a controlled fashion into the lumen of the needle, while the tip is plugged forcing material through the holes. A material can then be solidified by any one of the following including, but not limited to cooling, cross-linking by heat or exposure to ultraviolet radiation. In some embodiments, a material upon solidification is at least as compliant as the material into which it will be inserted. In some embodiments, holes are on the order of 100 microns in width or diameter as observed in the barbed region of the quill most directly correlated with decreased penetration force. In some embodiments, the holes are made at a distance from the tip of the needle that mimics the relative distance observed for a porcupine quill (e.g., 3-4 mm). In another preferred embodiment, the holes are made in the tapered portion of the needle.

In some embodiments, the morphology of protrusions can be imprint coated, laser cut, machined, or etched into a hypodermic needle. In some embodiments, the morphology can be achieved by molding one or a plurality of existing porcupine quills to create a negative mold. A negative mold can then be filled with a liquid material that upon solidification yields a positive cast of the porcupine quill.

Devices and methods described herein can be used in various applications including, but not limited to, medical devices, drilling, nailing, fishing, fastening, sewing, clothing manufacture, textiles, hair clips, holding devices, assembling layered systems, industrial adhesives, skin piercing (including ear piercing), shoemaking, or industrial puncturing devices.

In some embodiments, a device can be dimensioned and constructed for use as a needle, a microneedle array, a patch, a hook, a probe, a trocar, an implant, etc. A substrate can be compliant or non-compliant. In some embodiments, a substrate can be a tissue at a target site. Exemplary tissues includes, but are not limited to, skin, muscle, heart, spleen, liver, brain, intestine, stomach, gall bladder, blood vessels, fascia, dura, the eye, lips, tongue, mucosa, lungs, kidney, pancreas, and ears.

Provided devices and methods, in some embodiments, may be used in accessing sites in the body. In some embodiments, devices and methods described here can be used to insert into a site containing bodily fluids. Such devices and methods may be used in sampling bodily fluids or may further in processing for diagnostic purposes, acupuncture, tacking a film or mesh for treating hernia, ulcers, and burns, sealing internal or external wounds (suture/staple replacements/supplements).

In some embodiments, provided devices and methods can be used in applications of devices/tubes/monitoring systems/drug delivery devices to the skin or muscle or other tissues, preventing air leaks following lung resection procedures, delivering drugs, laparoscopically placing a tissue adhesive or buttress, obtaining vascular hemostasis, creating adhesion to the opthamalogic epithelium, and/or creating temporary surgical retraction.

In some embodiments, devices and methods described herein can be used in or as a mechanical adhesive. In addition or as an alternative, devices and methods described herein can be used as or in a delivery system and can release payloads after penetration to a substrate. In addition or as an alternative, devices and methods disclosed herein can be used for sampling and/or diagnostics.

### EXEMPLIFICATION

### Example 1

Materials and Methods

Material:

North American (specifically, Pacific Northwest) porcupine quills and African porcupine quills were purchased from Minute Bear Trading, USA. Fluorescein (sodium salt, dye content ˜70%, Aldrich), rhodamine B (dye content ˜90%, Sigma-Aldrich), ethanol (ACS reagent, ≧99.5%, 200 proof, Sigma-Aldrich), Sylgard® 184 silicone elastomer kit (Dow Corning, Corp., USA), UV-curable polyurethane acrylate (Minuta Tech., Korea), Irgacure 2959 (Ciba Specialty Chemicals Corporation), 18 gauge, 19 gauge, and 25 gauge 7/8 needles (Becton Dickinson Company), artificial human skin (SynDaver™ Labs), muscle tissue of domesticated fowl (Shaw's, Inc.), gelatin powder (Difco™, BD), sand paper (3M wet or dry sandpaper 413Q 400 and Norton MultiSand™, 60), cyanoacrylate glue (Loctite 495, Loctite Corp.), industrial razor blades (surgical carbon steel, single edged No. 9, VWR), polyether ether ketone (PEEK) hex nuts (Small Parts), silicone rubber film with backing adhesive (McMaster-Carr), pin mount stubs (25.4 mm in diameter, 9.5 mm in height, and 3.2 mm in pin diameter, Ted Pella, Inc.), 5 min and 60 min epoxy glues (ITW Performance Polymers) were used as received. The fresh porcine skin was purchased from a local butcher shop. The thin copper wire (˜0.2 mm in diameter) was extracted from electrical wire (Type AWM, RadioShack).

Penetration-Retraction Tests with Muscle Tissue and Gelatin Gel:

Penetration-retraction tests were performed with the mechanical tester (Model 5540, Instron Corporation). Only quills with a barbed region of 4 mm in length were selected for testing, as measured with a millimeter ruler and a dissecting optical microscope (SZ-6 PLUS, Cambridge Instruments). The muscle tissue was cut into specimens with 3-4 cm width, 2-3 cm length, and 4-5 mm thickness using a razor blade. The tissue specimens were mounted within the lower grips at the base of the mechanical tester. During fixation, care was taken not to excessively compress the tissue. After the specimen was fixed between the grips, the exposed excess tissue over the grips was cut with a blade, generating a flat tissue surface. The quill was fixed between the upper grips and the tip adjusted to contact the tissue surface. The quill was penetrated into the muscle tissue to the desired depth, typically 10 mm, at a rate of 1 mm/sec and was pulled out at a rate of 0.033 mm/sec to study how the barbs function during removal from tissue. For the duration of all experiments, the tissue was kept moist with phosphate buffered saline. Each quill was used for a single measurement. The experiments with gelatin gel, used as a control for a non-fibrous tissue were performed with an alternative set-up, as gelatin gel could not be gripped. Compression stage instead of the lower grips was used to fix the gelatin gel during measurements. In other words, gelatin gel was fixed onto the stage without compression. Considering this experimental set-up, we performed another test with muscle tissue by placing a thick section of tissue between lower grips without compression. To minimize any movement of muscle tissue, we prepared the chicken breast tissue that can fit with the available space between lower grips without compression. Gelatin gel was prepared with the same density as that of muscle tissue by dissolving gelatin powder into distilled water at 40° C. and letting it cool to room temperature. For the tissue and gelatin gel, the mean penetration force and mean pull-out force were measured from n=5 different samples.

Measurement of the Density of Muscle Tissue:

Tissue density was measured using previously described methods: Briefly, the tissue density was determined using a 25 ml glass pycnometer with the following equation (1).

dₛ=sd_{w}/(m_{pf}−mₜ+s) (1)

where dₛ is density of the tissue (g/mL), d_{w} is density of water (g/mL), s is weight of the dried tissue (g), m_{pf} is weight of pycnometer and water (g), and mₜ is weight of pycnometer, water and tissue. To obtain the weight of the dried tissue, the tissue had been dried in an oven at 60° C. until the weight plateaued after 7 days.

Preparation of the Stained Quills for Visualization During Adhesive Measurements:

Porcupine quills were immersed into 0.01% aqueous fluorescein or rhodamine B solution. After 1 h, quills were removed from the staining solution and washed thoroughly with water. The stained quills were dried overnight before use.

Penetration-Retraction Tests with Porcine Skin:

Fresh porcine skin was cut into specimens with 3-4 cm width and 3 cm length using a razor blade. For adhesive measurements, porcupine quills were inserted into porcine skin, vertically aligned within the lower grips, with a penetrating depth of 4 mm. The remainder of the test followed the procedure previously described for muscle tissue.

Characterization of the Mechanical Properties of Porcine Skin:

The specimen was cut into a dog bone shape (2 cm×5 cm). The length of skin for measurement (the distance between grips) was 6 mm. To prevent the porcine skin from slipping from the grips, the porcine skin was covered with sand paper (grit number: P60) except the measured area. The sand paper was tightly affixed to the porcine skin using cyanoacrylate glue and staples. A uniaxial tensile test was performed to measure mechanical properties of the prepared porcine skin using an eXpert 760 mechanical tester (ADMET, Inc.). The rate for the tensile test was 1 mm/min and the obtained data was fitted using the inverse Langevin model of finite elasticity.

Measurement of the Young's Modulus and Tensile Strengths for the Base and Tip of Porcupine Quills:

For measurements of the base, tensile tests were performed. In order to grip the base structure without crushing the ends, small steel needles (a tip part of 19 gauge needle) were inserted into both ends to maintain the cylindrical geometry. Copper wire and epoxy glue were then used on the exterior to prevent the samples from slipping from the grips. To accurately measure the Young's modulus of the tip, the top 1 mm near the apex was gripped with bottom grips leaving 2 mm exposed for measurement. We made the assumption that the cross-sectional diameter (˜0.30 mm) of the exposed 2 mm-long region between the grips remained constant. The prepared base and tip samples were mounted between lower grips and upper grips of the mechanical tester. The distance between lower grips and upper grips was 8 mm and 2 mm for base and tip, respectively. Measurements were performed at a rate of 1 mm/min for n=5 different samples.

Amino Acid Analysis:

Clean porcupine quills were cut and divided into 4 mm-length tip (i.e. only barbed region) and base, and 3 mg of each were gathered for analysis. Liquid phase hydrolysis of the samples was performed with 200 μL of 6N hydrochloric acid (HCl) added with 0.1% phenol at 110° C. for 24 h. After acid hydrolysis, the samples were dried for 1 h and then dissolved in Norleucine dilution buffer to a final volume as 20 or 40 mL. The final solution was thoroughly mixed with a vortexer and 50 μL was loaded into the analyzer (L-8800 Na Amino Acid Analyzer by Hitachi). The injected 50 μL contained 2.0 nmol of Norleucine as an internal standard.

Additional analysis was performed to obtain the concentration of cysteine which can be destroyed during hydrolysis with 6N HCl. Briefly, cysteine was oxidized to cysteic acid through incubating base and tip samples in 1.0 mL of performic acid at 4° C. overnight. The samples were then dried and prepared for amino acid content analysis as described above.

Buckling Resistance Tests:

To measure the buckling resistance of the shaft of the porcupine quill, 28 quills were randomly selected for uniaxial compression testing. The tip was trimmed slightly to provide a shaft with a near uniform cross sectional area and the resulting length and diameter were precisely measured with calipers. Quills were mounted in the mechanical tester between indented aluminum blocks to provide pivot connections at both ends of the quill. The mechanical tester was lowered at a rate of 100 mm/min and the resulting load was recorded. The critical load was determined by recording the load at buckling failure and plotted versus the slenderness ratio of the quill.

Surface Characterizations of the Quills:

The microstructures of the porcupine quills before and after penetration-retraction tests were examined with field-emission scanning electron microscope (FE-SEM, JEOL 5910) following a 30 nm-thick gold sputter coating. Light and fluorescence images were obtained with a Nikon Eclipse TE-2000-U microscope (Nikon Digital Sight DS-QiMC camera, Japan). The length of barbed region of each quill was examined with a dissecting optical microscope (SZ-6 PLUS, Cambridge Instruments) and optical digital images were obtained (IXY Digital, Canon, Japan).

Fabrication of Polyurethane (PU) Quills and Quill-Mimetic Needles:

Poly(dimethylsiloxane) (PDMS) pre-polymer was prepared by mixing the base material and curing agent in a 10:1 ratio. After vigorous mixing and degassing, PDMS molds of natural barbed and barbless quills were prepared by thermal curing at 70° C. overnight. To make quill-mimetic needle, a 25 gauge needle was inserted into the quills at this stage. After curing PDMS, the quill and needle were removed to produce PDMS molds. The polyurethane acrylate, which was mixed with 0.1% photo-initiator, was added into the PDMS molds. To fabricate a quill-mimetic needle, a 25 gauge needle was again inserted into the molds at this stage allowing the polyurethane to bond to the needle. Then, the samples were placed in a vacuum desiccator in the dark to degas the samples for 1-2 hours. The samples were then cured under UV (254 nm) for 90 min and removed from the molds.

Measurement of Penetration Force of PU Quills and Needles with Tissue:

A thick section of muscle tissue was prepared to fit with the available space between the lower grips of mechanical tester. The prepared tissue was placed between the grips without compression. The PU quill was fixed between the upper grips of mechanical tester and the tip adjusted to contact the tissue surface. The quill was penetrated into the muscle tissue to the desired depth, 4 mm, at a rate of 1 mm/sec. For the duration of all experiments, the tissue was kept moist with phosphate buffered saline. Each quill was used for a single measurement. The mean penetration force was measured from n=5 different samples.

The penetration force of quill-mimetic PU needle was examined with artificial skin (SynDaver Labs) that mimics the property of human skin. The fabricated PU needle was connected with a force gauge (Model FGV-5XY, Nidec-Shimpo Corp., Japan), and inserted manually into the skin tissue. The force gauge reads the required penetration force. Each needle was used at least 4 times. The mean penetration force was obtained from n=3 different samples.

Fabrication of Quill-Mimetic Patch with a Hexagonal Array of PU Quills:

The tip (5 mm-length) of natural quills was replicated with a hex nut base and arranged in a hexagonal array with a silicone backing layer using 60 min epoxy glue. Following generation of PDMS molds of barbed or barbless quills, we followed the same procedure descried previously to produce replica molded PU quills. The 7 PU barbed/barbless samples were then assembled with silicone backing layer. The hex base of PU quills allowed for simple alignment of a hexagonal array. To ensure that the array was stable, another backing layer was attached to the assembled sample using 5 min epoxy glue. All PU quills within the patch were perpendicular to the backing layer.

Measurement of Tissue Adhesion Force of Quill-Mimetic Patch:

A modification of ASTM F2258-05 was used to measure the tissue adhesion force of quill-mimetic patches. A flat section of muscle tissue was affixed using cyanoacrylate glue to test fixtures (i.e. pin mount stub with diameter of 25.4 mm). The prepared tissue sample was mounted within the lower grips at the base of the mechanical tester. The quill-mimetic patch was glued onto another fixture, and the prepared patch was fixed between the upper grips of mechanical tester. The tips of quills within the patch was adjusted to contact the tissue surface. The patch was penetrated into the muscle tissue to a depth of 4 mm at a rate of 1 mm/sec and was pulled out at a rate of 0.033 mm/sec to study how the barbs function during removal from tissue. For the duration of all experiments, the tissue was kept moist with phosphate buffered saline. The mean tissue adhesion force was measured from n=5 different samples.

Finite Element Analysis (FEA):

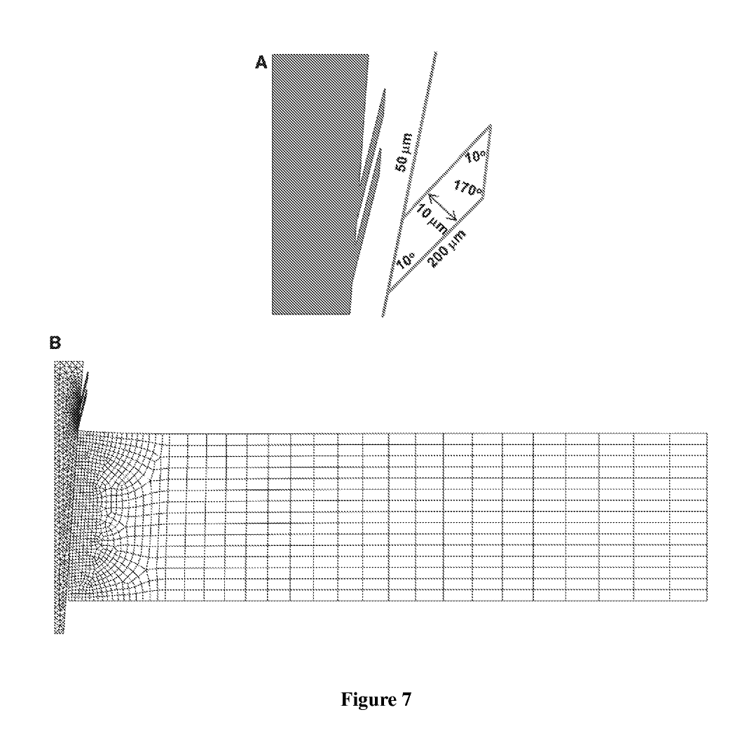

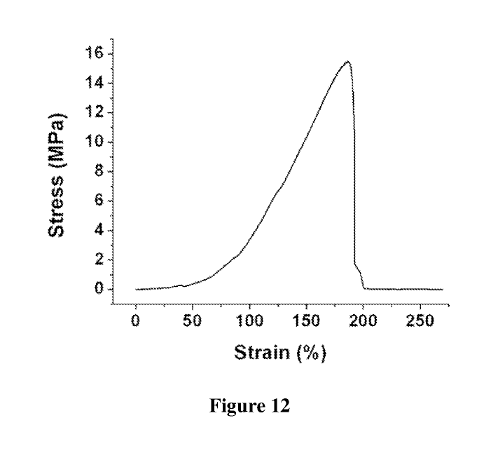

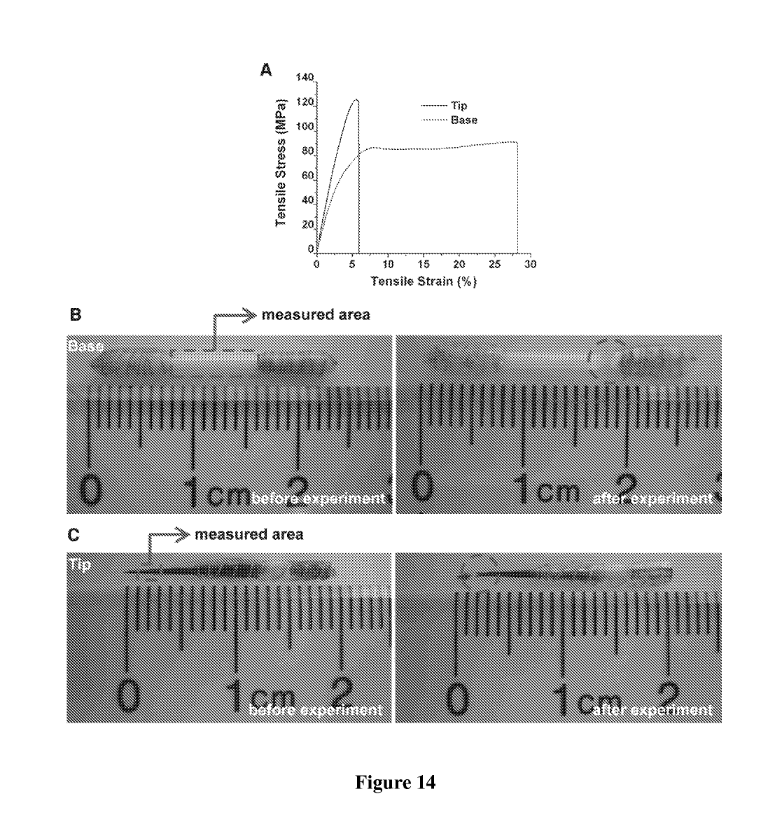

For the finite element simulation of the two-barbed quill penetrating through skin, we employed a two-dimensional approximation of the geometry with an initial mesh shown in FIG. 7. The quill component consists of 946 Abaqus CPE3 triangular elements, and the tissue consists of 982 Abaqus CPE4H hybrid quadrilateral elements. We model the quill and barbs as a linear elastic material with Young's modulus E=3.25 GPa and Poisson's ratio v=0.4 as determined from uniaxial tension experiments of quill tips (FIG. 14). To simulate the penetration of quills into the porcine skin, the mechanical response of porcine skin was analyzed and the tensile data was fitted to an inverse Langevin model for finite elasticity, and the rubbery modulus and network locking stretch of porcine skin was determined (FIG. 12). The rubbery modulus of porcine skin was 0.05˜0.28 MPa and its network locking stretch was 1.27˜2.35. The failure strength of porcine skin was 8.2˜15.4 MPa, which is similar to the values previously reported. For the finite element simulation, the skin is modeled as a non-linear incompressible material using the inverse Langevin model with an initial shear modulus μ=0.165 MPa and locking stretch λ_{L}=1.81. The simulation consists of two steps. To account for the already penetrated quill ahead of the section under consideration in this simulation, the quill is firstly translated to the right to pre-stress the tissue. Then in the second step, the quill is translated downward to slide across the tissue. This makes the model equivalent to considering a whole quill with only two barbs on its surface. Contact between the quill and the tissue is modeled as frictionless to model the wet environment encountered naturally.

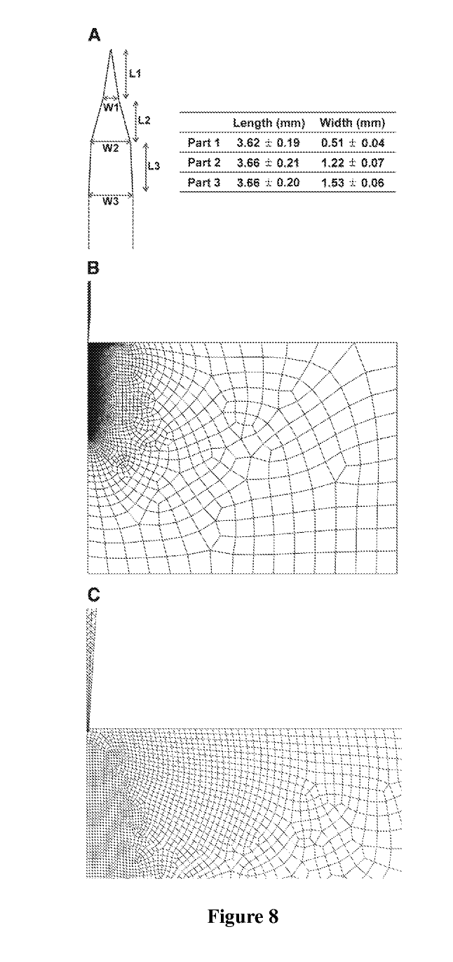

For the finite element simulation of the whole barbless quill penetration into porcine skin, we employed a two-dimensional approximation of the geometry, which is based on a natural quill, with an initial mesh shown in FIG. 8. The mesh of the quill consists of 257 Abaqus CPE4R quadrilateral and 201 Abaqus CPE3 triangular elements, also the tissue consists of 17422 Abaqus CPE4H hybrid quadrilateral elements. The displacement boundary conditions imposed on the quill included symmetry along the left edge, while the top edge is given a downward displacement of 10 mm to penetrate the quill into the tissue. The displacement boundary conditions on the tissue included nodes along the bottom and right edges that are pinned, while the top edge is traction free. To model the “tearing” of the tissue due to the penetration of the quill, a cohesive interaction between the elements of the tissue along the left edge, and a rigid surface (not shown) was defined. The cohesive interaction behaves elastically, with the same initial stiffness as the non-linear elasticity in the tissue, and then fails (loses load carrying capacity) at a maximum normal stress of 10 MPa (similar to experimental results (FIG. 12)). Currently, Abaqus/Standard doesn't support an axisymmetric cohesive interaction and failure, thus we have modeled the geometry as plane-strain to maintain the “tearing” property of penetration, which enables variation of material parameters to observe the relative differences between simulations. Contact between the quill and the tissue is modeled as frictionless to model the wet tissue environment that is naturally encountered.

Comparison Between Fibrous Tissue and Non-Fibrous Gelatin Gel to Examine Mechanical Interlocking of Tissue Fibers to Achieve Tissue Adhesion:

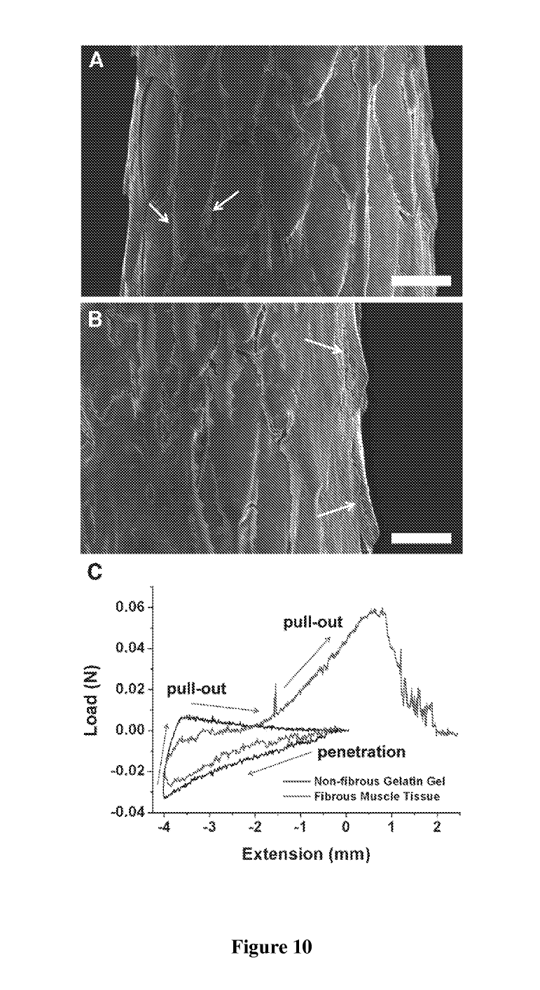

To examine if mechanical interlocking through hooking tissue fibers contributes to the tissue-holding force, we performed penetration-retraction tests using gelatin gel as a model of a non-fibrous tissue. To match the gelatin gel density with that of muscle tissue, we initially determined the fibrous component of muscle tissue to be 0.237±0.006 g/mL by obtaining the dry mass and wet volume (examined with a pycnometer) as previously described. Using the gelatin gel, we performed the tests with a penetrating depth of 4 mm. To prevent damage of the gelatin gel by gripping, the gel was placed on the mechanical tester without compression. This setup was repeated with muscle tissue accordingly to allow comparison of the gelatin and muscle data. As shown in FIG. 10C, the pull-out force generated with non-fibrous gelatin gel and a barbed quill was 0.009±0.003 N which was significantly lower than the force required to remove the 4 mm-penetrated porcupine quill from fibrous muscle tissue, 0.052±0.021 N. This data suggest that mechanical interlocking of tissue fibers by barbs is a significant factor to produce tissue adhesion.

Results and Discussion

The first step of biomimicry is to understand the mechanism that mediates the biological function. To this end, we have elucidated mechanisms for how the North American porcupine quill optimally interacts with tissue exhibiting both minimal penetration force and maximal pull-out force.

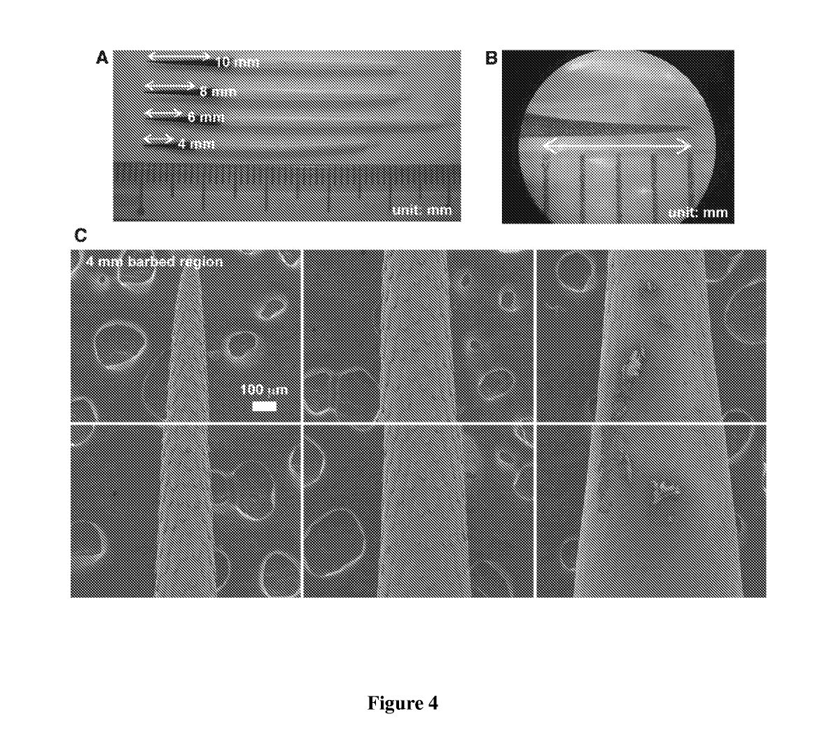

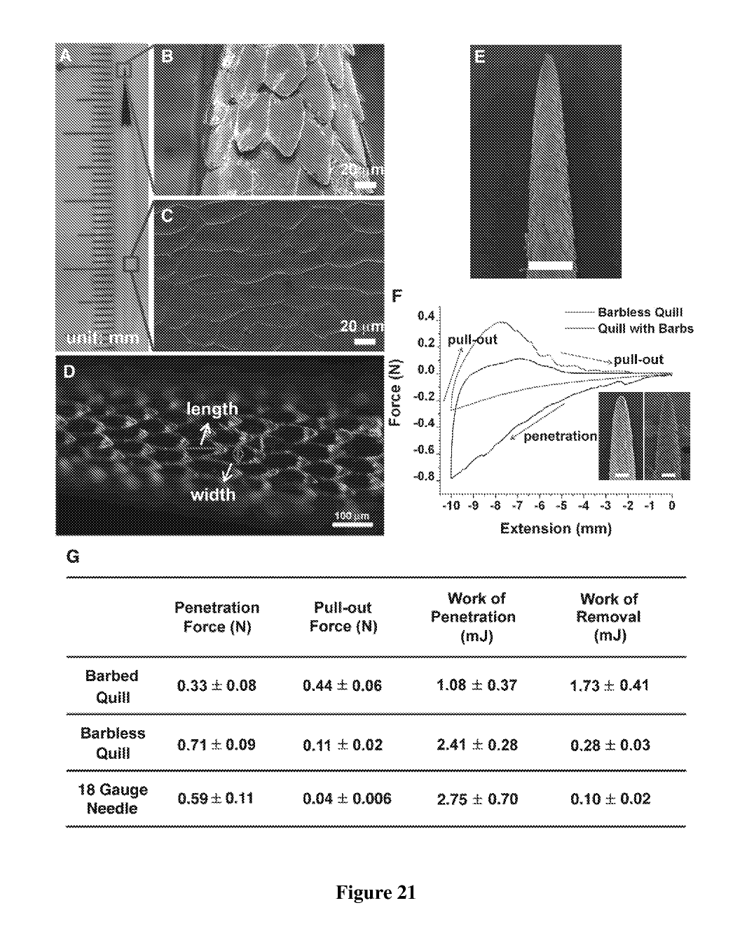

North American porcupine quills have two distinct regions that are demarcated by black (tip) and white (base) colors (FIG. 21A). While the conical black tip contains a layer of microscopic backward facing barbs on its surface, the cylindrical white base contains relatively smooth scale-like structures (FIG. 21B (Tip), C (Base)). As shown in FIG. 21D, barbs overlap slightly and the majority of barbs have dimensions ranging 100˜120 μm in length, with a maximum width of 35˜45 μm. There is 1˜5 μm space between the tip of each barb and the shaft of quill. At the apex of the tip, barbs are as short as 50˜70 μm as shown in FIGS. 21B and 21E whereas beyond 1 mm from the tip, the barbs are 170˜220 μm. Interestingly, as observed by light microscopy and confirmed by FE-SEM (FIG. 4), barbs exist on only a portion of the black tip and the length of the barbed region varies (typically in the range of 3-5 mm). Therefore, we standardized tests by using only quills with a barbed region of 4 mm.

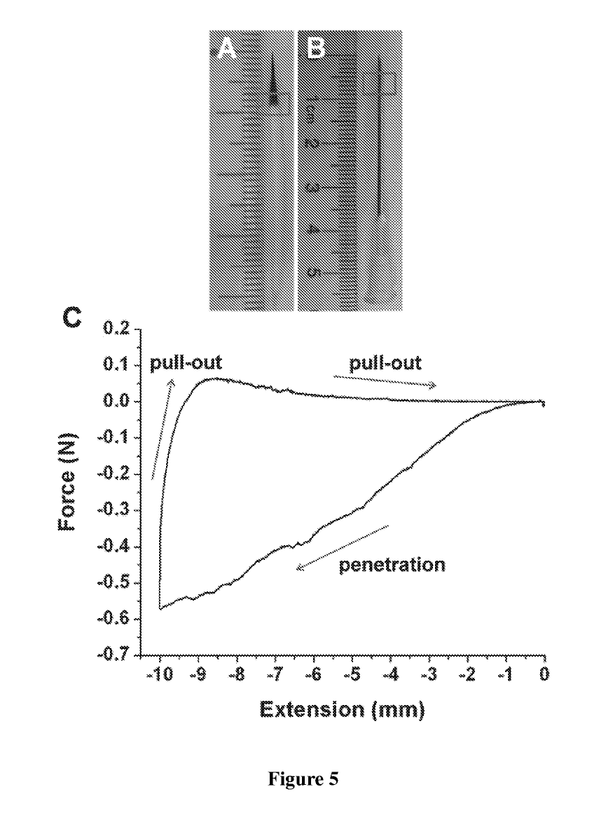

FIG. 21F shows the results of penetration-retraction tests including a barbless control quill whose barbs were carefully removed by gentle sanding. The penetrating depth into muscle tissue was set to 10 mm at a penetration velocity of 1 mm/s, and the force required for penetration was defined as the penetration force. Quills often pierce through skin into muscle tissue that may flinch and contract thereby pulling the quill deeper. In our experiments, the explanted muscle tissue was static, aside from when it was compressed during penetration followed by elastic relaxation as insertion force was removed. The maximum force required to remove the quill with respect to baseline was defined as pull-out force. Surprisingly, FIG. 21F shows that the quill with barbs required 54% less penetration force compared to the barbless quill. While the work of penetration for the barbed quill was 1.08±0.37 mJ, the barbless quill required 2.41±0.28 mJ (FIG. 21G). Regarding pull-out force, a quill with barbs required 0.44±0.06 N, the value for the barbless quill was 0.11±0.02 N. The work of removal for the barbed quill and barbless quill were 1.73±0.41 mJ and 0.28±0.03 mJ, respectively. Thus, it is clear that the presence of barbs significantly reduces the penetration force and leads to increased tissue-holding forces. Interestingly, the barbed quill requires less force and work to penetrate into tissue, compared to a medical hypodermic needle. Given that the average quill diameter was 1.161±0.114 mm, we examined the required force and work of penetration of an 18 gauge needle (diameter is 1.262±0.003 mm) into muscle tissue (FIG. 5). The average needle penetration force into muscle tissue was 0.59±0.11 N and the work of penetration was 2.75±0.70 mJ (FIG. 21G).

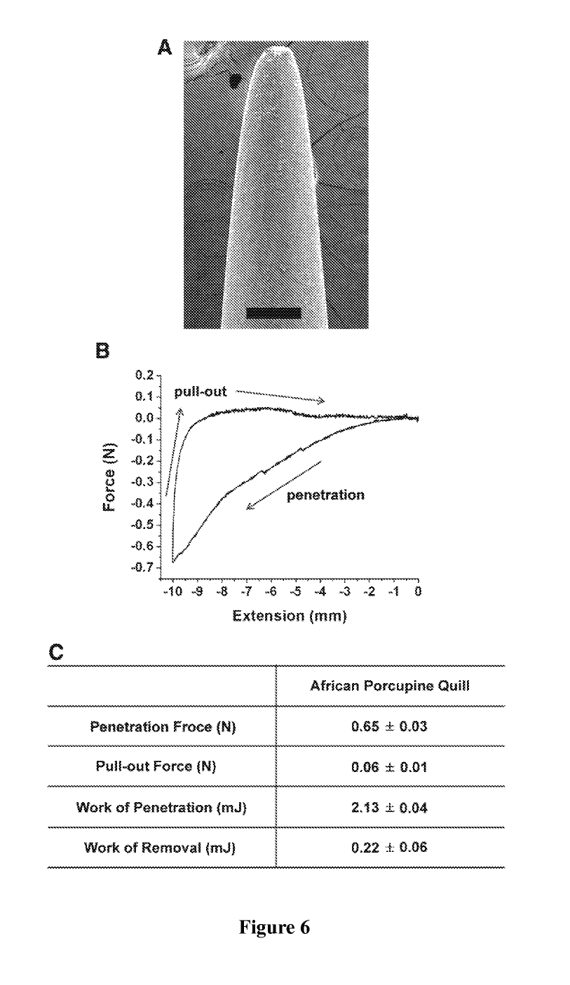

As an additional control for the presence of barbs, we performed penetration-retraction tests using the African porcupine quill that has a smooth surface as described in FIG. 6A: the work of penetration and work of removal were 2.13±0.04 mJ and 0.22±0.06 mJ, respectively (FIGS. 6B and C). These values and the profile of the force versus extension plot showed a similar trend to the barbless quill. Given that African porcupine quill and barbless North American quill showed similar penetration and pull-out behavior that was significantly different from that of the barbed North American quill, barbs appear essential for both reducing penetration force and generating tissue adhesion.

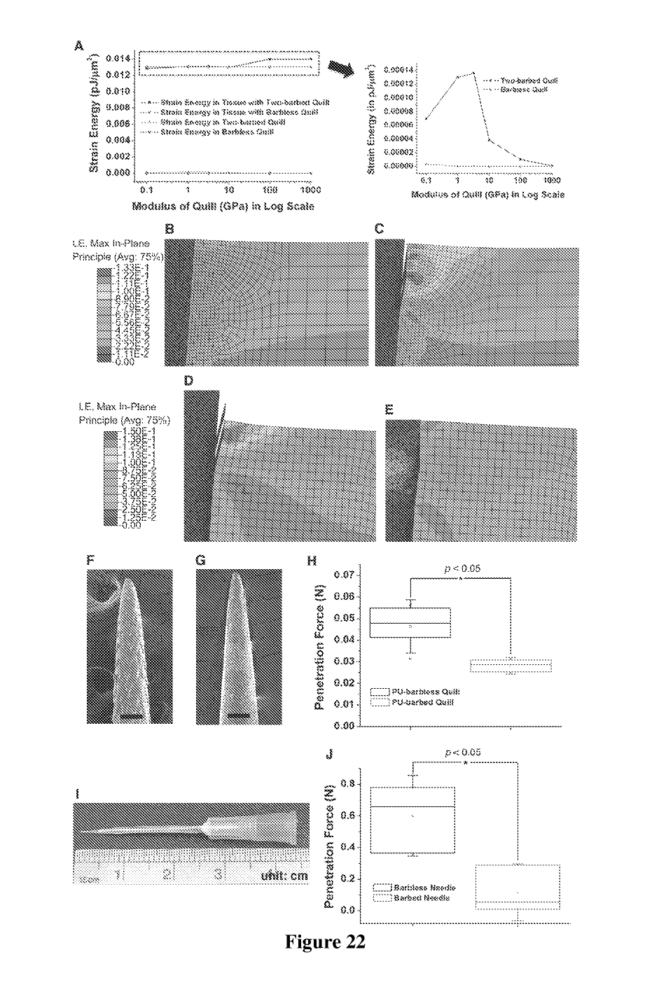

To understand the reduction of both penetration force and work of penetration required for the barbed quills, a simplified finite element model was developed (FIG. 7). We assume that the Work of Penetration=Quill Strain Energy+Tissue Strain Energy+Dissipation (including tearing of the tissue and may in practice include friction although this was not included in the model). By varying the stiffness of a simplified two-barbed quill component in a simulation (where the quill including its barbs exhibit uniform stiffness), we observed the strain energies absorbed by both the quill and tissue. Specifically, the strain energy in the natural quill (at 3.25 GPa) due to addition of barbs increases from 9.09 E-08 pJ/μm³ to 1.36 E-04 pJ/μm³ while the energy in the tissue increases from 0.01302 pJ/μm³ to 0.01310 pJ/μm³ (FIG. 22A). Given the negligible combined increase in strain energies in the quill and tissue, we postulate that the reduced work of penetration is facilitated by the reduction in dissipative energy such as tearing of tissue.

To penetrate into tissue, the quill must tear tissue at the tip as well as expand the hole circumferentially through stretching and tearing tissue fibers. To analyze the effect of barbs on this process, we looked at the strain distribution in the tissue using finite element analysis (FEA) for both barbless and two-barbed quills (FIGS. 22B and C). The analysis revealed that tissue is primarily stretched and deformed by the stress concentrations near the barbs. This happens regardless of the stiffness of the barbs (FIGS. 22D and E). We hypothesize the stress concentrations act to stretch and tear the tissue locally, reducing the need to deform the entire circumference of tissue surrounding the quill, and consequently reducing the penetration force. Thus the barbed features of the natural porcupine quill create an optimal surface to reduce penetration force through the creation of strategically placed stress concentrations. To confirm this hypothesis, we fabricated both barbed (non-deployable) and barbless polyurethane (PU) quills by replica molding (FIGS. 22F and G). The penetration force of both fabricated quills was examined by inserting them 4 mm into muscle tissue. While the penetration force of barbless PU quills was 0.046±0.010 N, the barbed PU quill required 35% less penetration force, 0.030±0.006 N (FIG. 22H). Additionally, the penetration force of natural barbed quill with the same muscle tissue was 0.043±0.013 N, which was not significantly different from the penetration force of PU barbed quill. Although the barbs of the PU quill cannot bend, the quill has many sharp points (i.e. barbs) where stress can be concentrated during penetration of the quill into tissue. Therefore, the experimental results with the fabricated PU quills support that stress concentration at barbs helps to reduce the penetration force of the natural porcupine quill. As the barbs have the same order of magnitude as muscle tissue fibers, which are in 50-100 μm, the concentrated stress at barbs likely help to cut tissue locally. This concept could potentially be used for the development of a novel medical needle with reduced penetration force. Towards this end, we fabricated a prototypic hypodermic needle that possessed the microscopic barbs of porcupine quills via replica molding. From the tests with a model for human skin, the PU-barbed needle showed 80% less penetration force, compared to the PU barbless needle (FIGS. 22I-J).

Another natural system that utilizes stress concentration to ease penetration is the mosquito's proboscis. Compared to the porcupine quill, it has a complicated mechanism utilizing three distinct needles that collectively ease penetration into tissue. The process involves first stretching the surface of an object with smooth labium, and then two jagged-shaped maxillas are inserted into the tissue, resulting in stress concentration between the two maxillas. Finally, the labrum, the blood drawing needle inserts into the object between the two maxillas. This operation of stretching and penetrating is repeated 30,000 times a second, gradually advancing the proboscis further into the tissue. In contrast to the mosquito that utilizes the coordinated movement of 5 structures to penetrate tissue, the porcupine quill is remarkably simple, requiring only its barbed geometry to reduce penetration force. In addition, the porcupine quill is unique in that it is geometrically optimized for both easy penetration and high tissue adhesion.

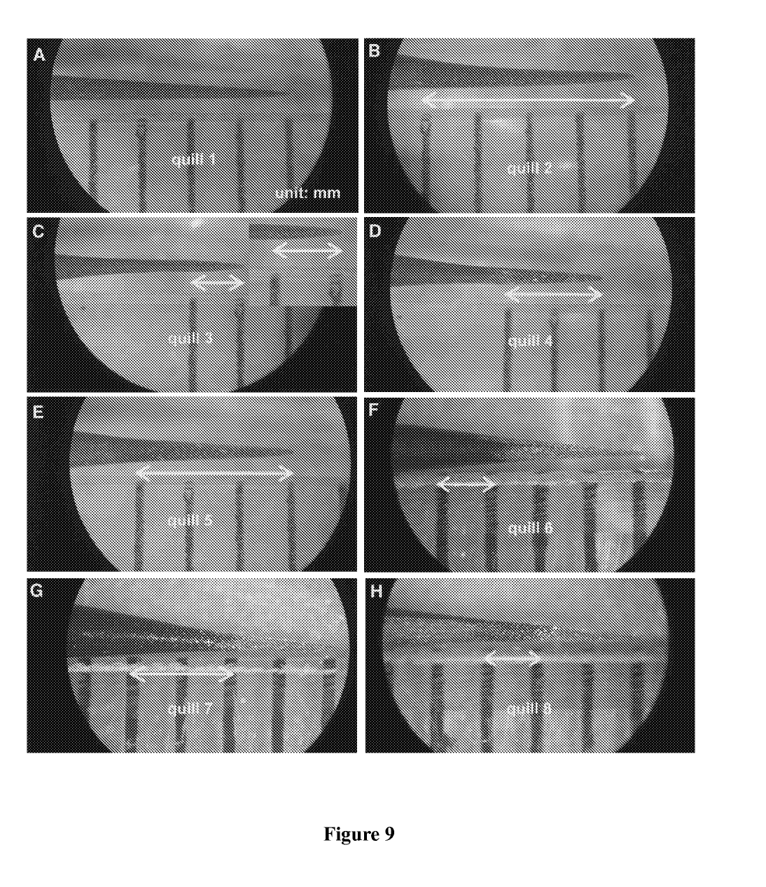

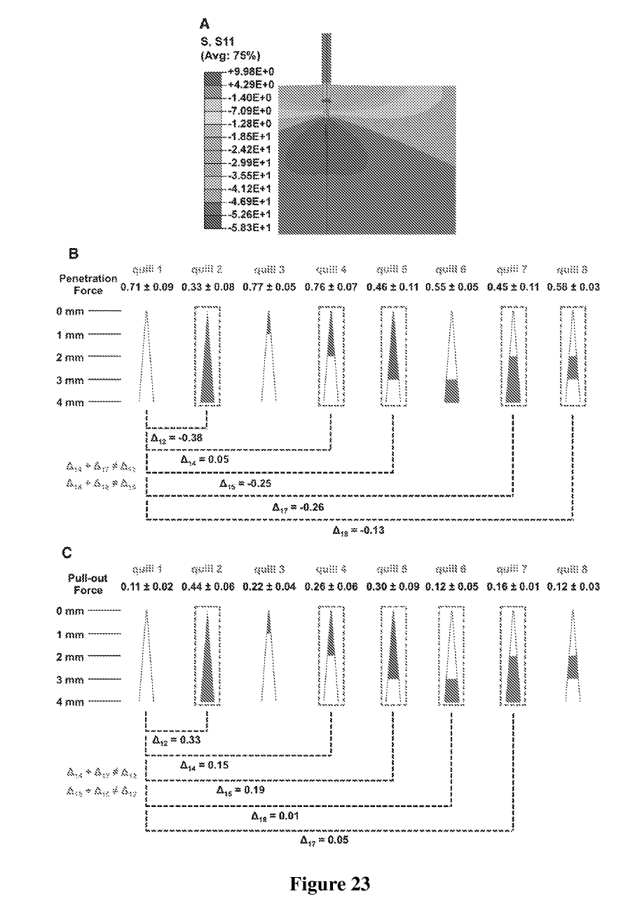

Further simplified modeling of the quill penetration using FEA revealed that the geometry of the quill tip is optimized for both easy passage through tissue and high resistance for removal (FIG. 23A). Specifically, upon insertion of a quill or needle into tissue, tensile and compressive ‘zones’ arise in the surrounding tissue. The quill has three geometrical transition zones as shown in FIG. 8. Tissue compression occurs tangential to the quill from the first transition zone, which is ˜3 mm from the apex of tip with a maximum at the second transition zone. This suggests that barbs closest to the first transition zone may experience the greatest interaction with tissue. To validate this, we used a sanding technique to produce quills that possessed barbs at specific regions (FIG. 9).

FIG. 23B shows that quill 1 requires 0.71±0.09 N to penetrate 10 mm into tissue whereas quill 2 requires 0.33±0.08 N (Δ₁₂=−0.38). Compared to the barbless quill, the force does not decrease if only the first 1 or 2 mm of barbed region at the tip of the quill is included (quills 3 and 4). However, when barbs in the 2-3 mm region are included, the penetration force significantly decreases. Quill 7, which has barbs only in the 2-4 mm region, resulted in a significant reduction of penetration force, ˜0.26 N. Additionally, the 2-3 mm (quill 8) or 3-4 mm (quill 6) barbed regions independently reduce the penetration force compared to the barbless quill (quill 1). These results suggest that the 2-4 mm barbed region close to the first transition zone of the quill is most important for reducing the penetration force. As the width of quill stem is larger at the first transition zone compared to the apex of the tip, the stress concentrated at the barbs within the transition zone likely facilitates cutting during penetration into tissue.

The presence of barbs contributes 0.33 N of pull-out force (comparing quills with a 4 mm barbed region, quill 2, to barbless quills, quill 1; Δ₁₂=0.33), 0.11 N was attributed to the first 1 mm of the barbed region (quill 3) at the tip (FIG. 23C). Comparing quill 3 and 5 (Δ₃₅=0.08) indicates that the 1-3 mm barbed region had less impact on pull-out force compared to the 1 mm region at the tip. Comparing the pull-out forces between quill 2 and 5 (Δ₂₅=0.14) suggests that the 1 mm region near the transition zone (at the base) is likely important. However, the presence of barbs solely in the 3-4 mm region (quill 6) or in the 2-4 mm region (quill 7) did not substantially increase pull-out force compared to the barbless quill. Furthermore, barbs in the 2-3 mm region alone did not increase the pull-out force. These data suggest that the barbs in different regions likely work cooperatively. Cooperativity is further supported by lack of additive effects (Δ₁₄+Δ₁₇≠Δ₁₂ and Δ₁₅+Δ₁₆≠Δ₁₂). Taken together, the results suggest that the first 1 mm region from the tip is the most important “individual” barbed region to increase pull-out force. However the 2-4 mm region significantly contributes to the increase in pull-out force by acting cooperatively with other barbed regions. Cooperativity may be a function of barb overlap where increased compressive force from tissue on barbs near the transition zone impacts barbs closer to the tip. Or barbs near the tip may experience different stresses due to tissue cut by the more proximal barbs. (see Table 1 below for a summary of work of removal for all quill preparations). Together this data suggests that the quill achieves adhesion by a mechanism that is more complex than simply hooking tissue fibers.

TABLE 1
Summarized work of penetration and work of removal
(from FIG. 23) obtained through penetration-retraction
tests with muscle tissue (n = 5). All the prepared quills
were inserted into the tissue to a depth of 10 mm.

| | Work of removal | Work of penetration |
|---|---|---|
| Classification of quills | from tissue (mJ) | into tissue (mJ) |
| quill 1 | 0.28 ± 0.03 | 2.41 ± 0.28 |
| quill 2 | 1.73 ± 0.41 | 1.08 ± 0.37 |
| quill 3 | 0.56 ± 0.07 | 2.72 ± 0.48 |
| quill 4 | 0.76 ± 0.28 | 2.32 ± 0.22 |
| quill 5 | 0.93 ± 0.26 | 1.35 ± 0.27 |
| quill 6 | 0.29 ± 0.06 | 1.82 ± 0.15 |
| quill 7 | 0.48 ± 0.09 | 1.51 ± 0.31 |
| quill 8 | 0.27 ± 0.06 | 1.90 ± 0.22 |

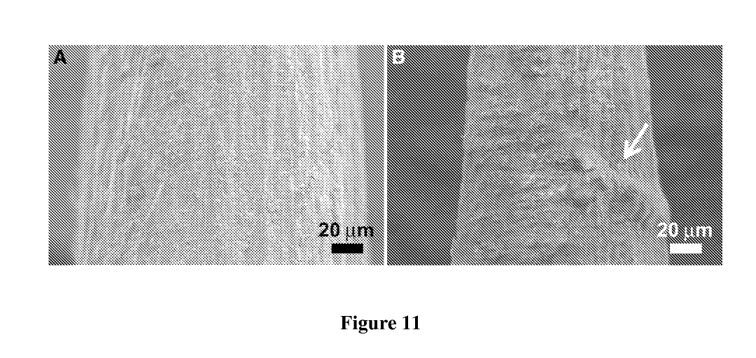

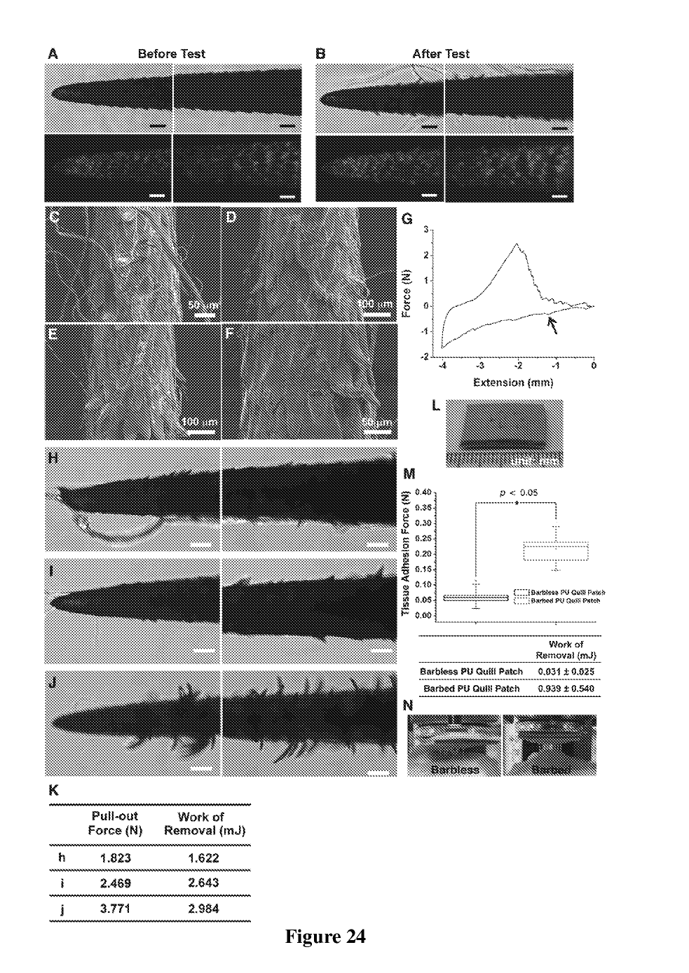

To examine how barbs generate mechanical adhesion with tissue, we examined quill removal from both fibrous tissue and a non-fibrous control in FIGS. 10 and 11. Interestingly, tissue fibers mechanically interlock under the barbs (FIG. 10B) suggesting barbs may be deployed or bent during removal from tissue. We postulated that such deployment of the barbs could increase tissue adhesion by projecting barbs radially away from the quill (thus increasing the apparent quill diameter) to significantly increase frictional resistance and promote further mechanical interlocking with tissue. Since in nature porcupine quills initially pierce the skin of an attacker, the ability of deployment or bending of barbs to contribute to tissue adhesion was tested with porcine skin, which has similar mechanical properties to human skin (FIG. 12). Bent barbs were clearly observed following penetration-retraction tests with bright field microscopy and fluorescent imaging followed by merging several images taken at different focal planes (FIGS. 24A and B). Through FE-SEM, the deployment or bending of barbs was observed with significant residual tissue adhered to the quill (FIGS. 24C-F). The pull-out force from the test with porcine skin was 2.36±0.83 N (FIG. 24G) while the work of removal was 2.34±0.68 mJ. Interestingly, we observed a direct correlation between the pull-out force and the number of bent barbs observed following removal of the quill from skin (FIGS. 24H-K). Reproducing the strong tissue adhesion property of the porcupine quill would be useful for the development of mechanically interlocking tissue adhesives. As a proof of concept, we fabricated a prototypic quill-mimetic patch that has a hexagonal array of 7 replica molded PU quills. (FIG. 24L). While the barbless PU quill patch showed minimal pull-out resistance (0.063±0.033 N), the barbed PU quill patch achieved significantly greater tissue adhesion (0.219±0.059 N, FIG. 24M). The work of removal for the barbed quill patch was >30× that of the barbless quill patch (FIG. 24M). As observed in FIG. 24N, the barbed quill array achieved significant interlocking with tissue whereas the barbless quill array achieved minimal interaction with tissue and thus could be easily removed.

The modulus of the quill may be optimized for penetration and adhesion as shown by the enlarged strain energy in the quill (FIG. 22A). When the quill is soft, it is easy to slide the quill through the tissue, but difficult to puncture tissue. Similarly, during pull-out the barbs may easily bend back flat against the stem, resulting in a low pull-out force. If the quill is too stiff, it strains tissue more by requiring greater tissue displacement to clear the protruding barbs, and the barbs cannot bend radially away from the quill. When the natural quill (3.25 GPa) is penetrated into tissue, the barbs likely bend slightly inward facilitating penetration. Furthermore, when the natural quill is pulled out of tissue, the force exerted by the tissue is sufficient to cause the barbs to project radially outward.

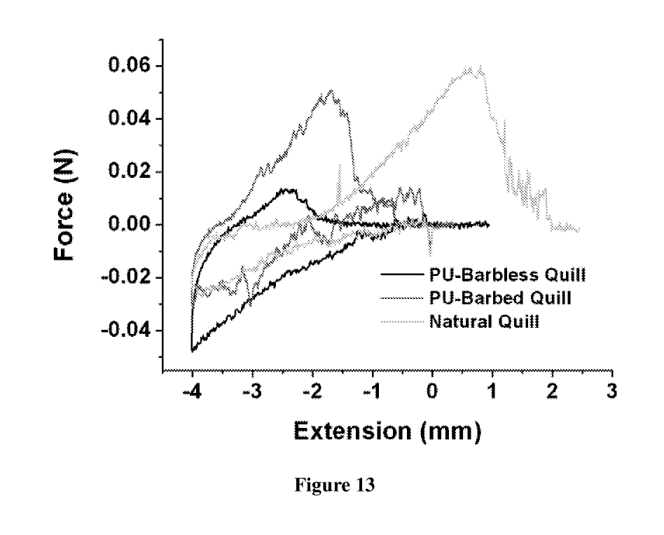

It is important to consider how the deployment of barbs affects penetration force and tissue adhesion. Our data suggests that the deployment of barbs is not critically important to reduce penetration force, as the penetration force of natural quills and PU barbed quills are similar (FIG. 13). However, deployment of barbs to achieve maximum adhesion is, important as shown by the significantly greater work required to remove a natural quill from tissue compared to a PU barbed quill (FIG. 13). Although the peak pull-out force is similar for the PU and natural quills, there are significant differences in the pull-out profile and work of removal. Specifically, natural quills with deployable barbs require 0.144±0.048 mJ for removal, compared to 0.053±0.023 mJ for non-deployable PU-barbed quills. The PU-barbed quill produces the maximum force after 2 mm of pull-out and then disengages the tissue completely at 4 mm of pull-out. However, the natural quill drags tissue for a relatively long displacement generating peak adhesion after it has been pulled out beyond 4 mm. The difference between natural quill and PU barbed quill is likely caused by the deployment of barbs. When the natural quill is pulled out, the barbs bend back, hook tissue underneath the bent barbs, and thus the quill remains engaged with tissue, stretching fibers even after it is pulled out beyond zero extension. However, the barbs of PU quills cannot bend, instead they cut tissue at higher tension when the quill is pulled out. In other words, the natural quill is able to stretch tissue maximally by using the bending of barbs, which may reduce cutting of tissue and likely grip more tissue fibers during pull-out.

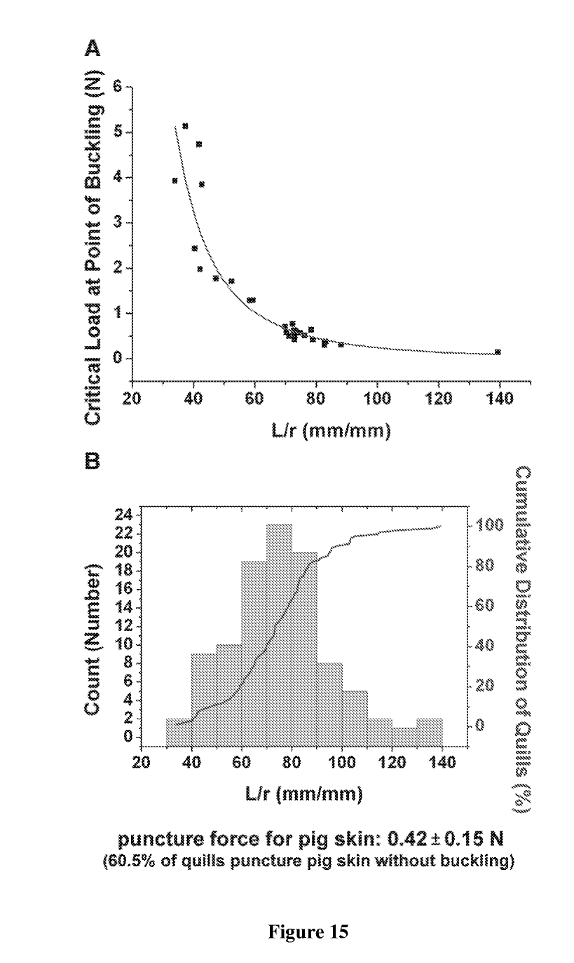

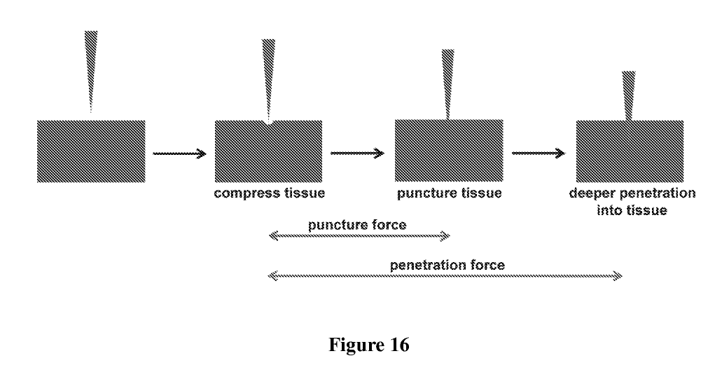

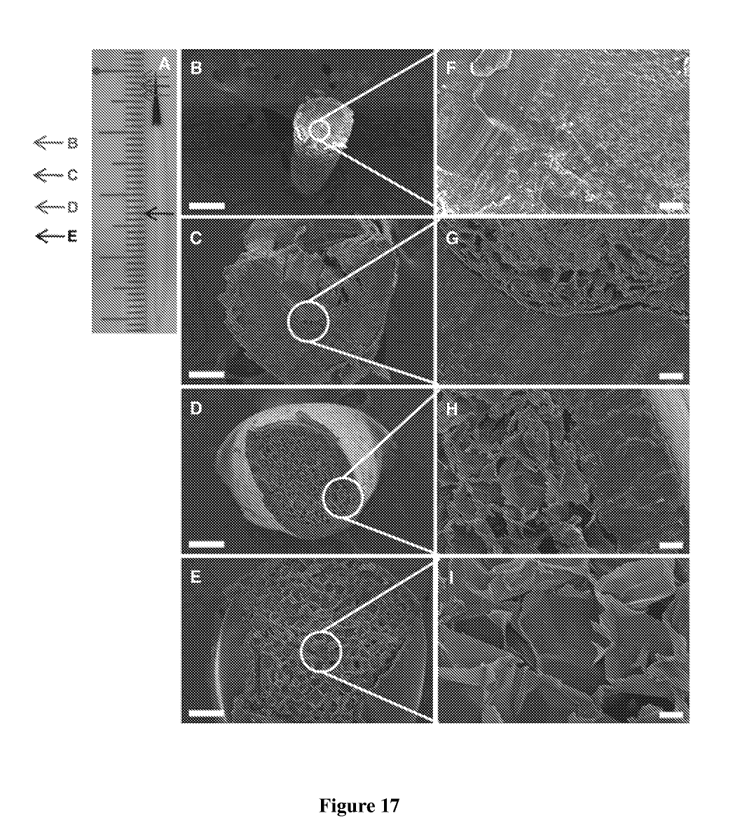

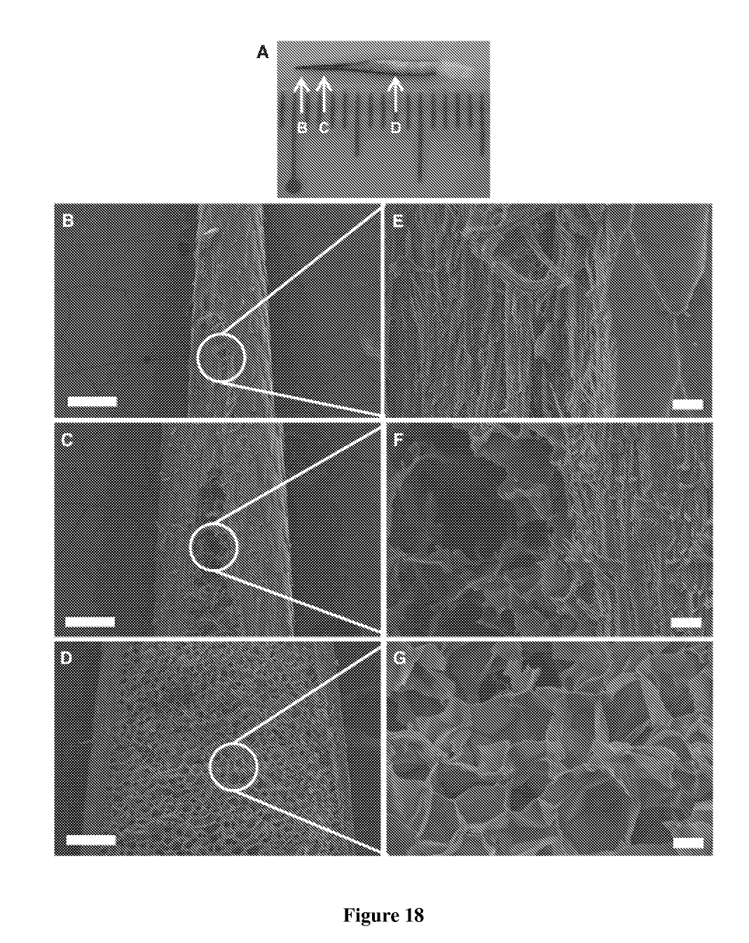

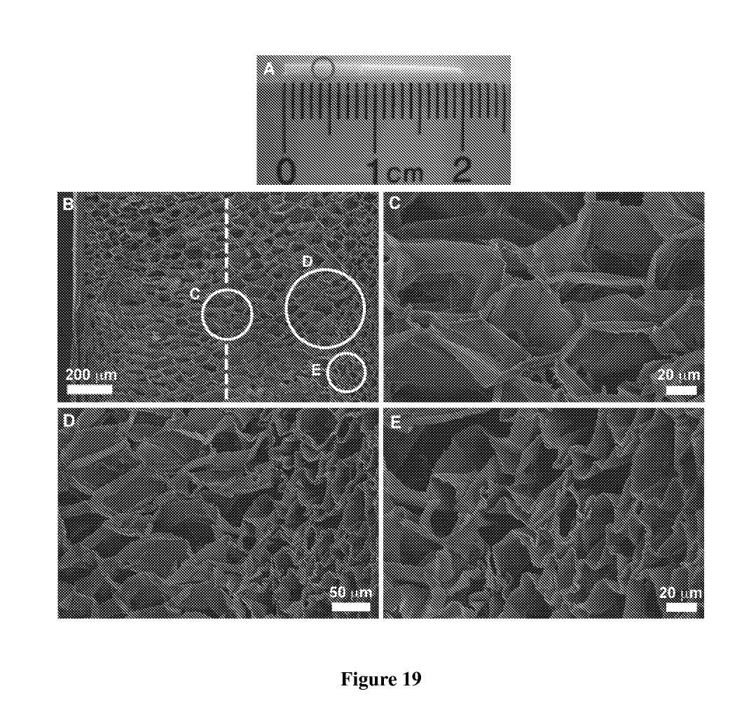

The efficient penetration of the porcupine quill into tissue is facilitated by the mechanical properties of the quill. In addition to Young's modulus, which is described in FIG. 14, the buckling resistance of the quill was investigated under uniaxial compression. Given that the tip is engaged with tissue during penetration, we considered the buckling resistance of the base of the quill with tip removed. As expected, the buckling resistance is a function of the quill slenderness ratio (length divided by radius) (FIG. 15). To examine the capacity of the porcupine's quills to resist buckling, we compared the buckling vs. slenderness ratio data with the cumulative distribution of quill slenderness ratios (FIG. 15). Theoretically, most quills (60.5%) can puncture skin without buckling: the puncture force was determined to be 0.42±0.15 N for the North American porcupine quill (shown as an arrow in FIG. 24G) and is defined as the force required to break through tissue before deep penetration (see FIG. 16 for the difference between puncture force and penetration force). The high buckling resistance is supported by the inner structure of the porcupine quill. FE-SEM shows that the apex of the tip is densely packed without pores, yet quickly transitions to a foam-filled tubular structure within the base, consistent with previous observations (FIG. 17). Interestingly, upon cutting a longitudinal section from tip to base, the apex of the tip was observed to have a fibrous uniaxial morphology (aligned along the length of the tip) (FIG. 18). Within the foam-filled tube (base), the cell size decreases radially from the center to the edge (FIG. 19), thus concentrating the material at the outer regions of the quill cross-section and increasing its cross-section moment of inertia. As previously described for cylinders with foam filled cores, the foam architecture likely increases buckling resistance during penetration of the quill into the flesh of a predator. Under uniaxial load, foam architectures act as an elastic foundation where the stress is the highest at the outer edge and decays radially inward to resist buckling.

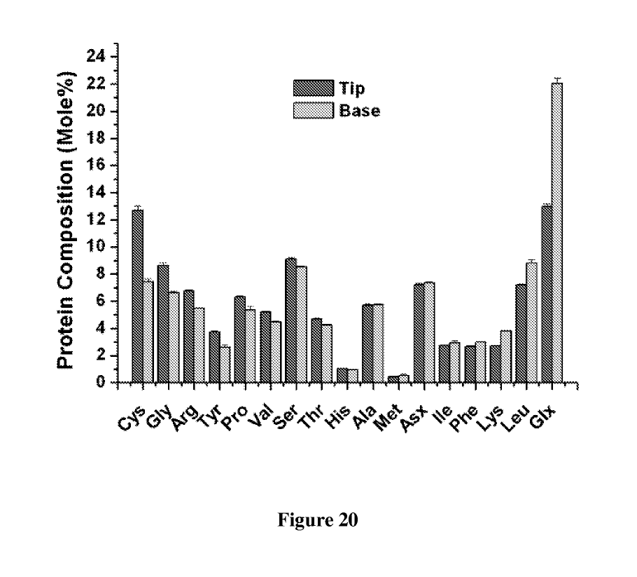

In addition to the porcupine quill's sharp tip and wide base with an inner foam core, a stiff tip likely aids in insertion into the flesh of predators by resisting buckling: the amino acid composition of the porcupine quill is dominated by cysteine, glycine, serine, and glutamine/glutamate. Interestingly, the porcupine quill tip contains significantly higher cysteine than the quill base, likely leading to an increased number of disulfide bridges that can confer increased strength through permanent and thermally stable cross-links (FIG. 20).

Herein we report how the North American porcupine quill is optimized for polar opposite functions including ease of penetration into tissue while retaining significant tissue adhesion force through the presence of backwards facing deployable barbs. Barbs located near the first transition zone exhibit the most substantial impact on minimizing the force required for penetration by facilitating ease of tissue fracture via the stress concentration at barbs, while barbs at the tip of the quill independently exhibit the greatest impact on tissue holding force. We have shown cooperation between barbs in the 0-2 mm and 2-4 mm regions that appear important to enhance pull-out forces. The tip of the porcupine quill is architecturally and chemically optimized for maximum stiffness to facilitate ease of penetration into tissue while the base's foam structure deems it light-weight, yet able to resist buckling during penetration into tissue.
1. U. Roze, The North American Porcupine. (Cornell University Press, Ithaca, ed. Second Edition, 2009).
2. J. F. V. Vincent, P. Owers, Mechanical design of hedgehog spines and porcupine quills. J. Zool. 210, 55 (September, 1986).
3. T. A. Vaughan, J. M. Ryan, N. J. Czaplewski, Mammalogy. (Saunders College Publishing, ed. Fourth Edition, 2000).
4. A. Kurta, Mammals of the Great Lakes Region. (The University of Michigan Press, ed. Revised Edition, 1995).
5. U. Roze, A facilitated release mechanism for quills of the North American porcupine (erethizon dorsatum). J. Mammal. 83, 381 (2002).
6. P. Forbes, The gecko's foot: Bio-inspiration: Engineering New Materials from Nature. (W. W. Norton & Company, Inc., New York, 2006).
7. F. Maier, A. Bornemann, Comparison of the muscle fiber diameter and satellite cell frequency in human muscle biopsies. Muscle Nerve 22, 578 (May, 1999).
8. S. Aoyagi, H. Izumi, M. Fukuda, Biodegradable polymer needle with various tip angles and consideration on insertion mechanism of mosquito's proboscis. Sens. Actuator A—Phys. 143, 20 (2008).
9. J. Ankersen, A. E. Birkbeck, R. D. Thomson, P. Vanezis, Puncture resistance and tensile strength of skin simulants. Proc. Inst. Mech. Eng. Part H—J. Eng. Med. 213, 493 (1999).
10. C. Edwards, R. Marks, Evaluation of biomechanical properties of human skin. Clin Dermatol 13, 375 (July-August, 1995).
11. G. N. Karam, L. J. Gibson, Biomimicking of animal quills and plant stems—natural cylindrical-shells with foam cores. Mater. Sci. Eng. C—Biomimetic Mater. Sens. Syst. 2, 113 (1994).
12. G. N. Karam, L. J. Gibson, Elastic buckling of cylindrical-shells with elastic cores .1. analysis. Int. J. Solids Struct. 32, 1259 (April-May, 1995).
13. G. N. Karam, L. J. Gibson, Elastic buckling of cylindrical-shells with elastic cores .2. experiments. Int. J. Solids Struct. 32, 1285 (April-May, 1995).
14. J. E. Bertram, J. M. Gosline, Functional design of horse hoof keratin: the modulation of mechanical properties through hydration effects. J Exp Biol 130, 121 (1987).
15. R. D. B. Fraser, T. P. MacRae, The Mechanical Properties of Biological Materials. J. F. V. Vincent, J. D. Currey, Eds., (Cambridge University Press, Cambridge, 1980).
16. M. Yang, J. D. Zahn, Microneedle insertion force reduction using vibratory actuation. Biomed. Microdevices 6, 177 (September, 2004).
17. G. R. DiResta et al., Measurement of brain tissue density using pycnometry. Acta Neurochir Suppl (Wien) 51, 34 (1990).
18. A. Cohen, A pade approximant to the inverse Langevin function. Rheol. Acta 30, 270 (May-June, 1991).
19. E. M. Arruda, M. C. Boyce, A three-dimensional constitutive model for the large stretch behavior of rubber elastic materials. J. Mech. Phys. Solids 41, 24 (1993).
20. J. A. Clark, J. C. Y. Cheng, K. S. Leung, Mechanical properties of normal skin and hypertrophic scars. Burns 22, 443 (September, 1996).
21. J. E. Sanders, B. S. Goldstein, D. F. Leotta, Skin-response to mechanical stress: adaptation rather than breakdown—a review of the literature. J. Rehabil. Res. Dev. 32, 214 (October, 1995).

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### OTHER EMBODIMENTS AND EQUIVALENTS

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A device for penetrating a substrate comprising
one or more tips, wherein the one or more tips are designed and constructed to initiate penetration by the device; and
one or more protrusions in a region adjacent to each tip.

2. The device of claim 1, wherein the one or more protrusions are constructed and arranged so that the required penetration force is reduced as compared with that observed for an otherwise identical device lacking the one or more protrusions.

3. The device of claim 1 or 2, wherein the one or more protrusions are constructed and arranged such that the required pull-out force is increased as compared with that observed for an otherwise identical device lacking the one or more protrusions.

4. The device of any one of the preceding claims, wherein the diameter of the penetration point of each tip is less than 120% of that observed for the otherwise identical device.

5. The device of any one of the preceding claims, wherein the one or more protrusions are located in a tapered region of each tip.

6. The device of any one of the preceding claims, wherein the substrate is compliant or non-compliant.

7. The device of any one of the preceding claims, wherein the substrate is tissue.

8. The device of any one of the preceding claims, wherein the one or more protrusions are spaced less than 1 cm, 5 mm, 500 microns, 100 microns, 10 microns or 1 micron apart.

9. The device of any one of the preceding claims, wherein the one or more protrusions are barb-shaped, hemisphere, pyramid, harpoon-shaped, triangle, conical, hook-shaped, oval or Y-shaped

10. The device of any one of the preceding claims, wherein the one or more protrusions have different shapes independently.

11. The device of any one of the preceding claims, wherein the one or more protrusions detach from the device.

12. The device of any one of the preceding claims, wherein the one or more protrusions comprise a bioerodible and/or biodegradable polymer.

13. The device of any one of the preceding claims, wherein the one or more protrusions have a length in a range of 50-250 μm or 1 μm-1 mm.

14. The device of any one of the preceding claims, wherein the one or more protrusions have a length in a range of 50-70 μm, 100-120 μm, or 170-220 μm.

15. The device of any one of the preceding claims, wherein the one or more protrusions have a maximum width of 35-45 μm, 50-250 μm or 1 μm-1 mm.

16. The device of any one of the preceding claims, wherein the one or more protrusions are arranged to face away from the tip so that, upon insertion, the one or more protrusions bend inward while, and upon pull-out, the one or more protrusions bend outward.

17. The device of any one of the preceding claims, wherein the one or more protrusions are arranged such that the one or more protrusions bend inward and act independently to reduce the required penetration force.

18. The device of any one of the preceding claims, wherein the region adjacent to each tip is about 2-4 mm.

19. The device of any one of the preceding claims, wherein the one or more protrusions are arranged such that the one or more protrusions bend outward and act cooperatively to increase the required pull-out force.

20. The device of any one of the preceding claims, wherein the region adjacent to each tip is about 2-4 mm.

21. The device of any one of the preceding claims, wherein the one or more protrusions are overlapped with one another such that the one or more protrusions work cooperatively to increase the required pull-out force.

22. The device of any one of the preceding claims, wherein the one or more protrusions are arranged such that the one or more protrusions bend outward and act independently to increase the required pull-out force.

23. The device of any one of the preceding claims, wherein the region adjacent to each tip is about 0-1 mm.

24. The device of any one of the preceding claims, wherein the one or more protrusions are arranged unidirectionally to face away from the tip.

25. The device of any one of the preceding claims, wherein the degree between the one or more protrusions and the surface of the one or more tips is in a range of 0-90 degrees, 1-60 degrees, or 1-20 degrees.

26. The device of any one of the preceding claims, wherein the one or more protrusions have an overlap of 1-50%, 5-30%, 10-20%, or up to 90% with one another.

27. The device of any one of the preceding claims, wherein the region adjacent to each tip is at a distance that mimics the relative distance observed for a porcupine quill.

28. The device of any one of the preceding claims, wherein the region adjacent is about 1-5 mm, 2-4 mm, or 3-4 mm away from each tip.

29. The device of any one of the preceding claims, wherein the device is characterized by the required penetration force being reduced to 10%, 25%, 35%, 75%, or 90%, as compared with that observed for the otherwise identical device.

30. The device of any one of the preceding claims, wherein the device is characterized by the required pull-out force required being increased to 1500%-125%, 400% or 200% as compared with that observed for the otherwise identical device.

31. The device of any one of the preceding claims, wherein the one or more protrusions comprise a material selected from the group of a polymer, a metal, a ceramic, and any combination thereof.

32. The device of any one of the preceding claims, wherein the one or more protrusions are characterized by a bending stiffness being 1-100 folds, 1-20 folds, or 1-10 folds greater as compared with that observed for the substrate.

33. The device of any one of the preceding claims, wherein the one or more protrusions comprise a shape memory material.

34. The device of claim 33, wherein the one or more protrusions are constructed and arranged so that upon increasing temperature by insertion into the substrate, the one or more protrusions deploy by reverting back to a shape memory annealed form.

35. The device of claim 33, wherein the device is characterized by the required pull-out force being further increased as compared with that observed for an otherwise identical device lacking the shape memory material.

36. The device of any one of claims 33-35, wherein the shape memory material is selected from the group consisting of shape memory alloys (SMA) and shape memory polymers (SMP), as well as shape memory ceramics, electroactive polymers (EAP), ferromagnetic SMAs, electrorheological (ER) compositions, magnetorheological (MR) compositions, dielectric elastomers, ionic polymer metal composites (IPMC), piezoelectric polymers, piezoelectric ceramics, and any combinations thereof.

37. The device of any of claims 33-36, wherein the shape memory material is nitinol.

38. The device of any one of the preceding claims, wherein the one or more tips are water-swellable.

39. The device of any one of the preceding claims, further comprising a body.

40. The device of claim 39, wherein the body comprises an elastomeric polymer selected from the group consisting of poly(caprolactone), acrylated star-poly(q-caprolactone-co-d,l-lactide), poly(dimethyl siloxane), polyurethane, poly(butadiene), poly(citrate), poly(glycerol sebacate) (PGS), and, PGS-polyurethane derivative, and any combination thereof.

41. The device of claim 39, wherein the body is non-swelling.

42. The device of claim 39, wherein the body is anti-adhesive or repellant.

43. The device of claim 39, wherein the body is a film, a sheet, a tape, a needle, an array, a hook, or a probe.

44. The device of any one of claims 39-43, wherein the body is connected with the one or more tips through one or more shafts.

45. The device of claim 44, wherein the one or more shafts comprise a biodegradable material so that the one or more shafts degrade to release the one or more tips from the body.

46. The device of any one of the preceding claims, wherein the device comprise a degradable/erodible material.

47. The device of any one of the preceding claims, wherein the one or more protrusions are covered by a coating of a degradable material so that the one or more protrusions are exposed after degradation of the coating.

48. The device of claims 46 and 47, wherein the degradable material is a polymer selected from the group consisting of polylactic acid, polyglycolic acid, polylactic-co-glycolic acid, polylactide, polyglycolide, poly(lactide-co-glycolide), polydioxanone, polycaprolactone, polycarbonates, polyorthoesters, polyamino acids, polyanhydrides, polyhydroxybutyrate, polyhydroxyvalyrate, polypropylene glycol-co-fumaric acid), polyhydroxyalkanoates, polyesters, polyanhydrides, polyphosphazenes, poly(alkylcyanoacrylates), biodegradable hydrogels, biodegradable polyurethane, poloxamers, polyarylates, amino-acid derived polymers, amino-acid-based polymers, particularly tyrosine-based polymers including tyrosine-based polycarbonate, polyarylates, poly(β-amino ester), and any combination thereof.

49. The device of any one of the preceding claims, wherein the one or more tips comprise a hypodermic needle.

50. The device of any one of the preceding claims, wherein the one or more tips each has at least one hole and at least one lumen.

51. The device of claim 50, wherein the at least one hole communicates between the lumen and an exterior.

52. The device of claims 50 and 51, wherein the at least one hole is arranged and constructed such that a liquid material can be introduced to pass through the lumen and contact a surface of the tip.

53. The device of claim 52, wherein the liquid material solidifies at the surface of the tip and forms the protrusion.

54. The device of claim 39, wherein the body is non-erodible.

55. The device of claim 39, wherein the one or more tips or the one or more protrusions are erodible or degradable, and wherein the body is more slowly erodible or degradable as compared with the one or more tips or the one or more protrusions.

56. The device of any one of the preceding claims, wherein the one or more tips are constructed and arranged into a needle, a microneedle array, a probe, a hook, or a trocar.

57. The device of any one of the preceding claims, further comprising an array of conical, pyramidal, cylindrical, or rectangular prism protrusions.

58. The device of any one of the preceding claims, wherein the one or more protrusions are fabricated by a technique selected from the group of laser cutting, dry etching, wet etching, imprint coating, molding, stamping, embossing, two-photon lithography, three dimensional printing, electrospinning, imprinting, interference lithography and any combination thereof.

59. The device of any one of the preceding claims, wherein the device is used in an application selected from the group consisting of accessing medical sites, sampling bodily fluids for diagnostics, acupuncture, tacking a film or mesh for treating hernia, ulcers, and burns, sealing internal or external wounds, preventing air leaks following lung resection procedures, delivery drugs, laparoscopically placing a tissue adhesive or buttress, obtaining vascular hemostasis, creating adhesion to the opthamalogic epithelium, and creating temporary surgical retraction.

60. The device of any one of the preceding claims, further comprising a payload.

61. The device of claim 60, wherein the payload comprise a bioactive agent.

62. The device of claim 60, wherein the payload is coated onto a surface of the one or more tips.

63. The device of claim 60, wherein the payload is incorporated into a lumen of the one or more tips.

64. The device of any one of claim 61, wherein the bioactive agent is selected from the group consisting of antiviral agent, adhesive, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, and penetration enhancer.

65. A method of using a device for penetrating a substrate comprising:
one or more tips, wherein the one or more tips are designed and constructed to initiate penetration by the device; and
one or more protrusions in a region adjacent to each of the one or more tips, the method comprising penetrating the substrate with the device.

66. The method of claim 65, wherein the substrate is a body or body part.

67. The method of claim 66, wherein the step of penetrating comprises inserting the device into a site containing bodily fluids.

68. The method of claim 67, further comprising sampling the bodily fluids.

69. The method of claim 68, further comprising processing the sample for diagnostic purposes, acupuncture, tacking a film or mesh for treating hernia, ulcers, burns, sealing internal and/or external wounds.

70. The method of claim 66, wherein the substrate is a body tissue selected from skin, muscle, heart, spleen, liver, brain, intestine, stomach, gall bladder, blood vessels, fascia, dura, the eye, lips, tongue, mucosa, lungs, kidney, pancreas, and ears.

71. A method of making a device for penetrating a substrate comprising:
one or more tips, wherein the one or more tips are designed and constructed to initiate penetration by the device; and
one or more protrusions in a region adjacent to each of the one or more tips.

72. In a method of designing/manufacturing a device for penetrating a compliant substrate, which device comprises one or more tips for initiating penetration, the improvement that comprises:
incorporating one or more protrusions in a region adjacent to the one or more tips.

73. The device of claim 72, wherein the one or more protrusions are constructed and arranged such that the required penetration force is reduced as compared with that observed for an otherwise identical device lacking the one or more protrusions.

74. The device of claim 72 or 73, wherein the one or more protrusions are constructed and arranged such that required pull-out force is increased as compared with that observed for an otherwise identical device lacking one or more protrusions.

75. In a device for penetrating a substrate, which device comprises one or more tips for initiating penetration, the improvement that comprises:
one or more protrusions in a region adjacent to the one or more tips.

76. The device of claim 75, wherein the one or more protrusions are constructed and arranged, such that the required penetration force is reduced as compared with that observed for an otherwise identical device lacking the one or more protrusions.

77. The device of claim 75 or 76, wherein the one or more protrusions are constructed and arranged such that required pull-out force is increased as compared with that observed for an otherwise identical device lacking the one or more protrusions.
